SEQUENCE LISTING (7 sequences)

SEQ ID NO: 1 - protein, 12 aa, Homo sapiens
MPHSFANLPLRF

SEQ ID NO: 2 - protein, 8 aa, Homo sapiens
VPNLPQRF

SEQ ID NO: 3 - protein, 35 aa, Bos taurus
SLTFEEVKDWAPKIKMNKPVVNKMPPSAANLPLRF

SEQ ID NO: 4 - protein, 28 aa, Bos taurus
AMAHLPLRLGKNREDSLSRWVPNLPQRF

SEQ ID NO: 5 - protein, 37 aa, Rattus rattus
SVTFQELKDWGAKKDIKMSPAPANKVPHSAANLPLRF

SEQ ID NO: 6 - protein, 18 aa, Rattus rattus
ANMEAGTMSHFPSLPQRF

SEQ ID NO: 7 - DNA, 1190 bp, Homo sapiens
ataaacattgggctgcacatagagacttaattttagatttagacaaaatggaaattatttcatcaaaactattcattttattgactttagccacttcaagcttgttaacatcaaacattttttgtgcagatgaattagtgatgtccaatcttcacagcaaagaaaattatgacaaatattctgagcctagaggatacccaaaaggggaaagaagcctcaattttgaggaattaaaagattggggaccaaaaaatgttattaagatgagtacacctgcagtcaataaaatgccacactccttcgccaacttgccattgagatttgggaggaacgttcaagaagaaagaagtgctggagcaacagccaacctgcctctgagatctggaagaaatatggaggtgagcctcgtgagacgtgttcctaacctgccccaaaggtttgggagaacaacaacagccaaaagtgtctgcaggatgctgagtgatttgtgtcaaggatccatgcattcaccatgtgccaatgacttattttactccatgacctgccagcaccaagaaatccagaatcccgatcaaaaacagtcaaggagactgctattcaagaaaatagatgatgcagaattgaaacaagaaaaataagaaacctggagcctgtccctaaagctgtggcctgtaatctacaaatggctctatagcgaagaccacacggaagagtagctacatacacttcatcagctatggatcatcaacggcaatttttccttgtcagtacagctataatagtatcttgaaagttgtaaaaaaattaaagcatatttgttacgtaaagttaaaatgatttttgtctgaataaaaaaaaagcattgcaaatgctttagaaatctctgataatggagagagagacagaggaccctcctcactaccctatataaaaatcattggcacagttacacttaataaaaaaaattaaacagaagagcaccctgaaaaacattatgatggaaattaaatagtatgccagaataacatggttgacaaataagtgaacaaggattaaaaatcacttacaaacgtgtttctgtacaccctttctatcgtgtcaaatgttaatgaatctgtgatcaattgaaatgtaaatgtctgtgtaaaactacaaaataaaaactcttagactttagggagaaaagaaaa

(19) United States
(12) Patent Application Publication
(10) Pub. No.: US 2016/0257735 A1
(43) Pub. Date: Sep. 8, 2016

(54) REGULATION OF NPVF GENE AND ITS PRODUCTS: A METHOD FOR MODULATING ENERGY IMBALANCE

(71) Applicants: Leslie P. Kozak, Olsztyn (PL); Julia Jaroslawska, Olsztyn (PL); Institute of Animal Reproduction and Food Research PAS, Olsztyn (PL)

(72) Inventors: Leslie P. Kozak, Olsztyn (PL); Julia Jaroslawska, Olsztyn (PL)

(73) Assignee: Institute of Animal Reproduction and Food Research PAS, Olsztyn (PL)

(21) Appl. No.: 14/878,318

(22) Filed: Oct. 8, 2015

(30) Foreign Application Priority Data
Mar. 2, 2015 (PL) P.411444

(51) Int. Cl. C07K 16/18; A61K 31/16; C12N 15/113; A61K 38/17

(52) U.S. Cl. CPC C07K 16/18; A61K 38/1709; A61K 31/16; C12N 15/113; C07K 2317/76; C12N 2310/14

(57) ABSTRACT

The invention relates to methods for modulating energy utilization by targeting the Npvf pathway. Inhibition of the Npvf pathway promotes energy utilization in a subject.

### RELATED APPLICATIONS

This application is a continuation of and claims the benefit under 35 U.S.C. §119 to Polish application number P.411444, filed Mar. 2, 2015. The entire contents of this application is hereby incorporated by reference in its entirety.

### BACKGROUND OF THE INVENTION

Reduced ambient temperature will increase thermogenesis and reduce obesity. However its long-term effectiveness as a strategy to reduce obesity has been questioned because of the expectation that increased energy expenditure for the cold environment will increase food intake, thereby neutralizing the weight reducing effects of the cool environment (1), a skepticism also associated with the effectiveness of physical activity as an anti-obesity strategy (2). This skepticism emerges from the adipostat hypothesis itself, which predicts that reductions in fat mass by cold stimulation will be compensated by increased food intake to maintain its adiposity index (3). On the other hand, studies on loss of fat mass by increasing thermogenesis with the chemical uncoupler dinitrophenol (DNP) showed that increased food intake does not necessarily occur (4). Therefore compensation as predicted by the adipostat model may also not occur in association with BAT thermogenesis. Since chemical uncoupling by DNP, or even activation of thermogenesis by adrenergic receptor agonists (5), are unregulated inductions of thermogenesis, compared to normal physiological mechanisms regulating body temperature, the problem of predicting the effectiveness of achieving energy homeostasis from food intake and endogenous energy reserves during cold exposure remains.

### SUMMARY OF THE INVENTION

The invention in some aspects is a method for promoting energy utilization in a subject, by administering to the subject an effective amount of a Npvf pathway inhibitor to promote energy utilization. The Npvf pathway inhibitor may be an Npvf pathway antagonist, such as an anti-RFRP3 antibody, an anti-RFRP1 antibody, or an antibody that binds to an RFRP Receptor. In some embodiments the Npvf pathway inhibitor is an expression inhibitor such as an inhibitory nucleic acid, e.g. an antisense oligonucleotide or a siRNA.

In some embodiments the Npvf pathway inhibitor is administered orally. In other embodiments the Npvf pathway inhibitor is administered by injection.

The method may further include the administration of a dopaminergic pathway agonist.

In other aspects the invention is method for treating a wasting disorder in a subject, by administering to the subject an effective amount of a Npvf pathway inducing agent to reduce energy utilization. The wasting disorder may be, for instance, anorexia nervosa.

In some embodiments the Npvf pathway inducing agent is exogenous Npvf. The exogenous Npvf may be, for instance, RFRP1 and/or RFRP3. In other embodiments the Npvf pathway inducing agent is an Npvf expression vector or an Npvf mRNA. In yet other embodiments the Npvf pathway inducing agent is an Npvf activator.

The Npvf pathway inducing agent, in some embodiments is an NPFF-R1 agonist. In other embodiments the Npvf pathway inducing agent is an NPFF-R2 agonist.

In some embodiments the Npvf pathway inducing agent is administered orally or by injection.

In other embodiments the method may further include the administration of a dopaminergic pathway antagonist.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. The details of one or more embodiments of the invention are set forth in the accompanying Detailed Description, Examples, Claims, and Figures. Other features, objects, and advantages of the invention will be apparent from the description and from the claims.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A-1G: Changes in endogenous substrate utilization and food intake associated with cold-induced thermogenesis in mice with variable levels of DIO. Diet-induced increase in body weight (A). Reduction in body weight during cold exposure (B). Changes in body weight (C), fat mass (D), and fat free mass (E) before and after 4 days at 4° C. Daily changes in food intake during 4 consecutive days at 4° C. (F). Correlations between daily increase in food consumed and internal body reserves mobilized per day (G). Data are expressed as mean±SEM. *, significant differences Data are expressed as mean±SEM. *, significant differences (t test, *, P<0.05; **, P<0.01; ***, P<0.005; ****, P<0.001).

FIGS. 2A-2D: Insulin and leptin resistance in wild-type B6 DIO mice. Changes in plasma free fatty acids 2A), insulin (B) and leptin (C) before and after 4 and 7 days at 4° C. in greater and lesser obese B6 mice. Correlation between fat mass and plasma leptin levels in DIO mice (D). Data are expressed as mean±SEM. *, significant differences (t test, *, P<0.05; **, P<0.01; ***, P<0.005).

FIGS. 3A-3E: The effects of leptin deficiency on the relative utilization of food intake and endogenous energy stores during cold-induced energy expenditure. Changes in body weight (A), fat mass (B), fat free mass (C), and food intake (D) determined before and after cold adaptation protocol in Lep−/−, Lep+/− and wild-type Lep+/+ mice. The rate of an increase in food intake per degree Celsius reduction in ambient temperature in mutant Lep−/− and control mice (E). Data are expressed as mean±SEM. *, significant differences between mice (t test, *, P<0.05; **, P<0.01; ***, P<0.005; ****, P<0.001).

FIGS. 4A-4D: The effects on endogenous substrate utilization and food intake in cold-exposed ucp1−/− and ucp1+/? mice with DIO. Changes in body weight (A), fat mass (B), fat free mass (C), and food intake (D) measured at normal ambient temperature (24° C.) and during the cold adaptation protocol. Data are expressed as mean±SEM. *, significant differences between mice (t test, *, P<0.05; **, P<0.01; ***, P<0.005; ****, P<0.001).

FIGS. 5A-5C: Cumulative energy utilization from food intake and endogenous energy stores in 3 models of obesity. Comparison of energy utilization from endogenous reserves and food intake in greater obese and lesser obese B6 mice (A), mutant Lep−/− and Lep+/? controls (B), and Ucp1−/− and Ucp1+/? controls (C).

FIGS. 6A-6B: Cold induced food intake returns to baseline when ambient temperature returns to 24° C. Changes in food intake (A) and body weight (B) in DIO B6 mice that were brought back to standard temperature after cold exposure.

FIGS. 7A-7E: Npvf gene is a hypothalamic biomarker of cold-activated thermogenesis. List of the genes that were similarly regulated upon cold exposure in Lep−/− and wild-type Lep+/+ mice (A). Verification of microarray data using qRT-PCR in Lep−/− and wild-type Lep+/+ mice (B). Cold-induced changes in Npvf mRNA expression in greater obese and lesser obese B6 mice (C). Ambient temperature-dependent expression of Npvf mRNA in hypothalamus of chow-fed wild-type B6 mice (D). Time-course of changes in the level of Npvf mRNA in hypothalamus under low temperature conditions (E). Data are expressed as mean±SEM. *, significant differences between mice (t test, *, P<0.05; **, P<0.01; ***, P<0.005; ****, P<0.001).

FIGS. 8A-8G. Changes in endogenous substrate utilization and food intake associated with cold-induced thermogenesis in mice with variable levels of DIO. Daily changes in food intake during the last week (week 16^{th}) before cold exposure (A). Time-course of changes in body weight during cold exposure (B). Body weight (C), fat mass (D), and fat free mass (E) before and after 7 days at 4° C. Daily changes in food intake during 7 consecutive days at 4° C. (F). Correlations between adiposity index determined before cold exposure and the change in daily energy consumption in the cold (G). Data are expressed as mean±SEM. *, significant differences (t test, *, P<0.05; **, P<0.01; ***, P<0.005; ****, P<0.001).

FIGS. 9A-9B: Cold-activated thermogenesis in DIO mice. The rate of an increase in food intake per degree Celsius reduction in ambient temperature in mutant Ucp1^{-/-} and control mice (A). Changes in the expression of genes associated with cold-activated thermogenesis in brown fat before and after 4 and 7 days at 4° C. in B6 mice with variable levels of DIO (B). Data are expressed as mean±SEM. *, significant differences (t test, *, P<0.05; **, P<0.01; *** , P<0.005; ****, P<0.001).

FIGS. 10A-10D: The expression of Npvf in the hypothalamus responds to reduced ambient temperature and time of exposure to the cold, but is not associated with the level of non-shivering thermogenesis in iBAT. Cold-induced changes in Npvf mRNA expression in mutant Ucp1−/− and normal control Ucp1+/? mice (A). Changes in the expression of Ucp1 mRNA in brown (B) and white (C) adipose tissue and Npvf mRNA in hypothalamus (D) in response to 7 days of either CL 316,243 or saline administration at 29° C. in B6 mice. Data are expressed as mean±SEM. * , significant differences (t test, *, P<0.05; **, P<0.01; ***, P<0.005; ****, P<0.001).

FIGS. 11A-11B: Changes in the expression of genes encoding for thermogenic genes in iBAT (A) and iWAT (B) before and after 4 and 7 days at 4° C. in DIO B6 mice. Data are expressed as mean±SEM. *, significant differences (t test, P<0.05; **, P<0.01; ***, P<0.005; ****, P<0.001).

FIGS. 12A-12B: Changes in the expression of genes encoding neuropeptides associated with feeding behavior in hypothalamus before and after 4 and 7 days at 4° C. in DIO B6 mice (A) and in mutant Lep−/− and control wild-type Lep+/+ mice before and after the cold acclimatization protocol (B). Data are expressed as mean±SEM. *, significant differences (t test, *, P<0.05; **, P<0.01; ***, P<0.005).

### DETAILED DESCRIPTION OF THE INVENTION

Activation of thermogenesis in response to cold exposure initiates increased food intake and utilization of endogenous fuel reserves. However, when a subject is exposed to a cold environment it is unknown whether a physiological decision is made to use endogenous energy reserves or to increase food intake and whether this decision is influenced by the obese state of the subject. It has been discovered according to the invention that cold-induced thermogenesis is preferentially fueled by oxidation of fat reserves in subjects with environmental obesity and by food intake in lean subjects. Importantly, this decision to increase or decrease food intake to fuel thermogenesis upon cold exposure is independent of leptin action and brown fat thermogenesis.

In cold-exposed animals increased thermogenesis is associated with increased feeding, but is not accompanied by a gain of weight (6). Coordinated increases in thermogenesis and food intake during cold exposure are controlled by signaling events in the hypothalamus that are undefined. The contribution of selective neuro-hormone systems such as NPY or TRH in the regulation of cold-activated thermogenesis and feeding behavior has been extensively studied using pharmacologic approaches (13, 14) or animal knockout models (15-17). However, neither of these approaches identifies a critical molecule or describes signaling events that accounts for central mechanisms controlling energy availability and utilization under cold conditions. It has been discovered according to the invention that cold exposure causes a suppression of Npvf neuropeptide precursor mRNA levels in the hypothalamus of three models of obesity. This response is independent of food intake, endogenous fuel utilization or leptin or insulin regulation. Upon a return to room temperature following cold exposure Npvf mRNA levels are restored to those of mice kept at room temperature. To our knowledge Npvf is the only transcriptional target in hypothalamus known to be selectively regulated by changes in ambient temperature.

Adapting to a cold environment or increasing physical activity results in increased energy expenditure. The energy to fuel the increase in energy expenditure comes either from endogenous fuel reserves or from increased food intake. If it comes from endogenous fuel reserves, then the obese condition is reduced. The data presented herein shows that during cold-induced thermogenesis the fuel in obese subjects comes from endogenous fat stores, whereas in lean subjects it comes from food intake. Since these behavioral processes are controlled by the central nervous system, the determination of how the brain controls this behavior and thus the identification of therapeutic agents to optimize the utilization of fat stores to reduce obesity or alternatively to stimulate food intake and minimize fat utilization in conditions like anorexia nervosa have been studied and are described herein.

Since the balance between the 2 sources of energy (increased food intake and utilization of endogenous fuel reserves) can influence the obese state, the relationships between food intake and endogenous substrate utilization in 3 models of obesity was studied. Upon cold exposure C57BL/6J+/+(wild-type B6) and Ucp1−/− mice with diet-induced obesity (DIO) burn endogenous fat in direct proportion to their fat reserves and food intake is inversely related to fat mass, whereas leptin-deficient and wild-type B6 mice fed a chow diet depend almost solely on increased food intake. In an analysis of gene expression in the hypothalamus we have found that the most robust change in gene expression was associated with the suppression of Npvf precursor mRNA in the hypothalamus when mice are exposed to the cold. In rodents, Npvf mRNA encodes two peptides, RFRP-1 and RFRP-3 and is detected specifically in a region between the dorsomedial and ventromedial hypothalamic nuclei. A change in Npvf mRNA has not been previously associated with food intake, fat metabolism or thermogenesis or other forms of energy expenditure. The change in Npvf mRNA is similar in lean normal weight animals, in animals with diet-induced obesity and animals with inactive brown fat. In each case it is associated with cold-activated thermogenesis.

Thus, in some aspects the invention is a method for promoting energy utilization in a subject, by administering to the subject an effective amount of a Npvf pathway inhibitor to promote energy utilization. In other aspects the invention is a method for treating a wasting disorder in a subject by administering to the subject an effective amount of a Npvf pathway inducing agent to reduce energy utilization. Npvf (neuropeptide VF precursor) is a nucleic acid which encodes RFamide related peptides (RFRPs) including at least two peptides, RFRP1 and RFRP3.

Manipulation of the Npvf pathway can be used as described herein to regulate thermogenesis without cold induction in a manner that is independent of nutritional status. For instance, induced expression of Npvf precursor mRNA results in increased production of the encoded peptides, RFRP1 and/or RFRP3. These peptides bind to their G protein coupled receptors NPFFR (neuropeptide FF receptor), including NPFF-R1 and NPFF-R2 to regulate thermogenesis. Additionally, introduction of synthetic peptides or mimetics, agonists and antagonists by injection or ingestion or by alterations in the expression and processing of the Npvf gene products will activate or inhibit their biological functions.

An Npvf pathway inhibitor as used herein refers to a compound that interrupts the activity or expression of a component of the Npvf pathway. Components of the Npvf pathway include but are not limited to Npvf, RFRP and NPFF-R. The existence of a novel mammalian RFamide peptide gene was identified through analyzing the human genome and cDNA sequences in the GenBank/EMBL database. Through isolation of human, bovine, rat and mouse cDNAs by RT-PCR, three mature peptides (RFRP-1, -2 and -3) were generated in the bovine and human gene. In rodents, only RFRP-1 and -3 were generated. In mammals, mature RFRP-1 and RFRP-3 were purified from human, bovine and rat hypothalamic tissue. Distribution of RFRPs were analyzed in various tissues of several mammals using immunohistochemistry and in situ hybridization, these studies have so far indicated that RFRPs neurons in mammals are predominantly located between the ventromedial (VMN) and the dorsomedial (DMN) hypothalamic nuclei with fibre projections throughout the central nervous system. Identification of RFRP-1 and -3 receptors was reported using studies in transfected CHO cell lines following treatment with synthetic RFRPs. CHO cell lines transfected with a cDNA encoding a rat orphan 7TM GPCR (named OT7T022, later renamed GPR147) responded specifically to RFRP-1 and -3. GPR147 is identical to NPFFR1, which is a GPCR that specifically binds to and is activated by another RFamide peptide, neuropeptide FF (NPFF). Quantitative RT-PCR showed highest levels of GPR147 mRNA expressed in the rat hypothalamus with moderate levels in the thalamus, medulla oblongata, testis and eyes. In situ analysis revealed strong signals of GPR147 mRNA within paraventricular hypothalamic nucleus (PVN), specifically within the lateral portion and in the periventricular hypothalamic nucleus (PerVN). Another GPCR receptor, refered to as HLWAR177 or GPR74 (which corresponds to NPFFR2) also exhibited a response to RFRPs, but the activation was weak. GPR74 mRNA is expressed in various regions of the brain with high levels in the cingulate gyrus, placenta and adipose tissue and moderate levels in the pituitary, amygdala, hippocampus, hypothalamus and nucleus accumbens.

The human Npvf gene (also designated RFRP (RFamide-related peptide) gene or C7orf9 gene), is responsible for encoding three small neuropeptides designated RFRP-1 (NPSF), RFRP-2 and RFRP-3 (NPVF). The homologous gene in rodents encodes only two functional neuropeptides: RFRP-1 (NPSF) and RFRP-3 (NPVF). RFamide-related peptides constitute a large family of neuropeptides in a wide range of species that are known to play a role in neurotransmission, neuromodulation, cardioexcitation and control of muscle contraction. RFRP-1 and RFRP-3 are widely expressed in the retina and in fetal and adult brain, including the forebrain, hypothalamus, thalamus, midbrain, pons and medulla oblongata. The biological actions of these peptides are mediated via distinct but related surface receptors; RFRP-1 and RFRP-3 are preferential ligands of RFRP receptor (RFRPR; also termed NPFF1R or GPR147), whereas NPFF and NPAF bind and activate NPFF receptor (NPFFR; also termed NPFF2R). Thus, NPFF-R which includes NPFF1R and NPFF2R is equivalently referred to as a RFRP receptor (RFRPR) and GPR147. Npvf pathway inhibitors include but are not limited to Npvf pathway antagonists and Npvf pathway expression inhibitors. A wide variety of Npvf pathway inhibitors are commercially available and useful in the methods and products described herein. They include, for example, small molecule inhibitors such as NPFF-R antagonists such as RF9, BIBP3226, antibodies that bind to and block the activity of a component of the Npvf pathway, such as RFRP or NPFF-R, or an inhibitory nucleic acid, such as an antisense oligonucleotide or a siRNA.

An Npvf pathway antagonist as used herein is a molecule that blocks the activity of 1 or more components of the Npvf pathway. For instance, Npvf pathway antagonists include RFRP antagonists and NPFF-R antagonists. RFRP is an RF amide-related peptide derived from a FMRFamide-related peptide precursor (Npvf), which is cleaved to generate neuropeptide RFRP-1 (also referred to as NPSF), neuropeptide RFRP-2 and neuropeptide RFRP-3 (also referred to as NPVF). human RFRP-1 has the sequence MPHSFANLPLRF-NH(2) (SEQ ID NO: 1). human RFRP-3 has the sequence VPNLPQRF-NH(2) (SEQ ID NO: 2). Other RFRP-1 and -3 sequences include bovine (SLTFEEVKDWAPKIKMNKPVVNKMPPSAANLPLRF (SEQ ID NO: 3)) and (AMAHLPLRLGKNREDSLSRWVPNLPQRF (SEQ ID NO:4)) and rat (SVTFQELKDWGAKKDIKMSPAPANKVPHSAANLPLRF (SEQ ID NO: 5)) and (ANMEAGTMSHFPSLPQRF (SEQ ID NO: 6)) respectively.

RFRP antagonists include, for instance, antibodies that bind to RFRP and block the activity of RFRP. An anti-RFRP antibody, for instance, is an antibody which binds specifically with a RFRP such as RFRP-1 or RFRP-3 and interferes with RFRP activity. Anti-RFRP antibodies are commercially available from companies such as Thermo Fisher Scientific, Inc. (PAS-25175); Lifespan Biosciences (LS-C144065 LS-C144066 LS-C144067 LS-C144051); Biorbyt (orb35548 orb2444 orb4978); St John's Laboratory (STJ49444); Atlas Antibodies (HPA041733); Santa Cruz Biotechnology (sc-99158 sc-32374 sc-98572 sc-67009); Genway (GWB-DB8DE0) Enzo Life Sciences (BML-FA1155); EMD Millipore (AB15348); and Abgent. Santa Cruz antibodies to RFRP-3 have the following catalog numbers: sc-32377 and sc-32380. Santa Cruz antibodies to RFRP-1 have the following catalog numbers: sc-67010, sc-67009, sc-32379, sc-32374, and sc-32375. Other antibodies against RFRP may be generated and fall within the compounds useful according to the invention.

NPFF-R antagonists are compounds that functionally antagonize the NPFF receptor by interrupting the receptor ligand interaction and thus disrupting the receptor signaling. An example of an NPFF-R antagonist is RF9 (adamantylcarbonyl-arginyl-phenylalaninamide). The antagonist RF9 is selective for NPFF receptors, but does not distinguish between the NPFF1 and NPFF2 subtypes. Another example of an NPFF-R antagonist is BIBP3226, which is anorexigenic Y1 receptor ligand. BIBP3226 has been shown to bind to NPFFR1.

The Npvf inhibitor may also be an expression inhibitor. An Npvf expression inhibitor, as used herein, refers to a compound that can reduce expression levels of a component of the Npvf pathway in a subject. These inhibitors include but are not limited to inhibitory nucleic acids. The inhibitory nucleic acid may be, for instance, a siRNA or an antisense molecule that inhibits expression of a Npvf pathway mRNA or protein. The nucleic acid sequences of the components of the Npvf pathway are well known in the art. See for instance, Homo sapiens RFRP mRNA for RFamide-related peptide precursor, complete cds GenBank: AB040290.1; Homo sapiens neuropeptide NPVF precursor, mRNA, complete cds GenBank: AF330057.1; and Homo sapiens C7orf9 hypothetical protein mRNA, complete cds GenBank: AF440392.1. Human RFRP mRNA for RFamide-related peptide precursor, (complete cds) is found at accession number: AB040290.1, referencing Hinuma et al Nat. Cell Biol. 2 (10), 703-708 (2000). RFamide-related peptide receptor is found at accession number: NP_001092117.1 (peptide sequence). The inhibitory nucleic acids may be designed using routine methods in the art.

The Npvf pathway expression inhibitor may be a siRNA to Npvf, for instance. siRNAs are commercially available and can also be custom ordered from Dharmacon. The nucleotide sequences of Npvf molecules are well known in the art and can be used by one of skill in the art using art recognized techniques in combination with the guidance set forth herein to produce the appropriate siRNA molecules. Nucleic acid sequences for human Npvf can be found for instance as Homo sapiens neuropeptide VF precursor (NPVF), mRNA NCBI Reference Sequence: NM_022150.3. An exemplary nucleic acid sequence is:

| (SEQ ID NO: 7) | |
|---|---|
| 1 | ataaacattg ggctgcacat agagacttaa ttttagattt agacaaaatg gaaattattt |
| 61 | catcaaaact attcatttta ttgactttag ccacttcaag cttgttaaca tcaaacattt |
| 121 | tttgtgcaga tgaattagtg atgtccaatc ttcacagcaa agaaaattat gacaaatatt |
| 181 | ctgagcctag aggataccca aaaggggaaa gaagcctcaa ttttgaggaa ttaaaagatt |
| 241 | ggggaccaaa aaatgttatt aagatgagta cacctgcagt caataaaatg ccacactcct |
| 301 | tcgccaactt gccattgaga tttgggagga acgttcaaga agaaagaagt gctggagcaa |
| 361 | cagccaacct gcctctgaga tctggaagaa atatggaggt gagcctcgtg agacgtgttc |
| 421 | ctaacctgcc ccaaaggttt gggagaacaa caacagccaa aagtgtctgc aggatgctga |
| 481 | gtgatttgtg tcaaggatcc atgcattcac catgtgccaa tgacttattt tactccatga |
| 541 | cctgccagca ccaagaaatc cagaatcccg atcaaaaaca gtcaaggaga ctgctattca |
| 601 | agaaaataga tgatgcagaa ttgaaacaag aaaaataaga aacctggagc ctgtccctaa |
| 661 | agctgtggcc tgtaatctac aaatggctct atagcgaaga ccacacggaa gagtagctac |
| 721 | atacacttca tcagctatgg atcatcaacg gcaatttttc cttgtcagta cagctataat |
| 781 | agtatcttga aagttgtaaa aaaattaaag catatttgtt acgtaaagtt aaaatgattt |
| 841 | ttgtctgaat aaaaaaaaag cattgcaaat gctttagaaa tctctgataa tggagagaga |
| 901 | gacagaggac cctcctcact accctatata aaaatcattg gcacagttac acttaataaa |
| 961 | aaaaattaaa cagaagagca ccctgaaaaa cattatgatg gaaattaaat agtatgccag |
| 1021 | aataacatgg ttgacaaata agtgaacaag gattaaaaat cacttacaaa cgtgtttctg |
| 1081 | tacacccttt ctatcgtgtc aaatgttaat gaatctgtga tcaattgaaa tgtaaatgtc |
| 1141 | tgtgtaaaac tacaaaataa aaactcttag actttaggga gaaaagaaaa |

A Npvf pathway inhibitory nucleic acid typically causes specific gene knockdown, while avoiding off-target effects. Various strategies for gene knockdown known in the art can be used to inhibit gene expression. For example, gene knockdown strategies may be used that make use of RNA interference (RNAi) and/or microRNA (miRNA) pathways including small interfering RNA (siRNA), short hairpin RNA (shRNA), double-stranded RNA (dsRNA), miRNAs, knockout plasmids, including CRISPR/Cas9 based plasmids and other small interfering nucleic acid-based molecules known in the art. In one embodiment, vector-based RNAi modalities (e.g., shRNA expression constructs) are used to reduce expression of a gene (e.g., a target nucleic acid such as a Npvf pathway nucleic acid) in a cell. In some embodiments, therapeutic compositions of the invention comprise an isolated plasmid vector (e.g., any isolated plasmid vector known in the art or disclosed herein) that expresses a small interfering nucleic acid such as an shRNA. The isolated plasmid may comprise a specific promoter operably linked to a gene encoding the small interfering nucleic acid. In some cases, the isolated plasmid vector is packaged in a virus capable of infecting the individual. Exemplary viruses include adenovirus, retrovirus, lentivirus, adeno-associated virus, and others that are known in the art and disclosed herein.

A broad range of RNAi-based modalities could be employed to inhibit expression of a gene in a cell, such as siRNA-based oligonucleotides and/or altered siRNA-based oligonucleotides. Altered siRNA based oligonucleotides are those modified to alter potency, target affinity, safety profile and/or stability, for example, to render them resistant or partially resistant to intracellular degradation. Modifications, such as phosphorothioates, for example, can be made to oligonucleotides to increase resistance to nuclease degradation, binding affinity and/or uptake. In addition, hydrophobization and bioconjugation enhances siRNA delivery and targeting and siRNAs with ribo-difluorotoluyl nucleotides maintain gene silencing activity. siRNAs with amide-linked oligoribonucleosides have been generated that are more resistant to S1 nuclease degradation than unmodified siRNAs. In addition, modification of siRNAs at the 2′-sugar position and phosphodiester linkage confers improved serum stability without loss of efficacy. Other molecules that can be used to inhibit expression of a gene (e.g., a Npvf pathway gene) include sense and antisense nucleic acids (single or double stranded), ribozymes, peptides, DNAzymes, peptide nucleic acids (PNAs), triple helix forming oligonucleotides, antibodies, and aptamers and modified form(s) thereof directed to sequences in gene(s), RNA transcripts, or proteins.

Triple helix approaches have also been investigated for sequence-specific gene suppression. Triple helix forming oligonucleotides have been found in some cases to bind in a sequence-specific manner. Similarly, peptide nucleic acids have been shown to inhibit gene expression. Minor-groove binding polyamides can bind in a sequence-specific manner to DNA targets and hence may represent useful small molecules for suppression at the DNA level. In addition, suppression has been obtained by interference at the protein level using dominant negative mutant peptides and antibodies. The diverse array of suppression strategies that can be employed includes the use of DNA and/or RNA aptamers that can be selected to target a protein of interest (e.g, components of the Npvf pathway).

Other inhibitor molecules that can be used include sense and antisense nucleic acids (single or double stranded). Antisense nucleic acids include modified or unmodified RNA, DNA, or mixed polymer nucleic acids, and primarily function by specifically binding to matching sequences resulting in modulation of peptide synthesis. Antisense nucleic acid binds to target RNA by Watson Crick base-pairing and blocks gene expression by preventing ribosomal translation of the bound sequences either by steric blocking or by activating RNase H enzyme. Antisense molecules may also alter protein synthesis by interfering with RNA processing or transport from the nucleus into the cytoplasm.

As used herein, the term “antisense nucleic acid” describes a nucleic acid that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript. Those skilled in the art will recognize that the exact length of the antisense oligonucleotide and its degree of complementarity with its target will depend upon the specific target selected, including the sequence of the target and the particular bases which comprise that sequence.

In some embodiments the inhibitory nucleic acid of the invention is 100% identical to the nucleic acid target. In other embodiments it is at least 99%, 95%, 90%, 85%, 80%, 75%, 70%, or 50% identical to the nucleic acid target. The term “percent identical” refers to sequence identity between two nucleotide sequences. Percent identity can be determined by comparing a position in each sequence which may be aligned for purposes of comparison. Expression as a percentage of identity refers to a function of the number of identical amino acids or nucleic acids at positions shared by the compared sequences. Various alignment algorithms and/or programs may be used, including FASTA, BLAST, or ENTREZ-FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

An inhibitory nucleic acid useful in the invention will generally be designed to have partial or complete complementarity with one or more target genes (i.e., complementarity with one or more transcripts of components of the Npvf pathway). The target gene may be a gene derived from the cell, an endogenous gene, a transgene, or a gene of a pathogen which is present in the cell after infection thereof. Depending on the particular target gene, the nature of the inhibitory nucleic acid and the level of expression of inhibitory nucleic acid (e.g. depending on copy number, promoter strength) the procedure may provide partial or complete loss of function for the target gene. Quantitation of gene expression in a cell may show similar amounts of inhibition at the level of accumulation of target mRNA or translation of target protein.

“Inhibition of gene expression” refers to the absence or observable decrease in the level of protein and/or mRNA product from a target gene. “Specificity” refers to the ability to inhibit the target gene without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). Depending on the assay, quantitation of the amount of gene expression allows one to determine a degree of inhibition which is greater than 10%, 33%, 50%, 90%, 95% or 99% as compared to a cell not treated according to the present invention. As an example, the efficiency of inhibition may be determined by assessing the amount of gene product in the cell: mRNA may be detected with a hybridization probe having a nucleotide sequence outside the region used for the inhibitory nucleic acid, or translated polypeptide may be detected with an antibody raised against the polypeptide sequence of that region.

Additionally, several siRNAs to Npvf pathway components are available commercially. For instance siRNA to human and mouse RFRP are available from Santa Cruz Biotechnology (sc-44797 and sc-44798). siRNA directed to human NPVF are also available commercially from QIAGEN. These include for example predesigned siRNA directed against human NPVF (NM_022150) available as product number SI00132972, SI00132979, or SI00132993. OriGene also has RNAi products in human, mouse, rat for Npvf. RFRP CRISPR/Cas9 Knockout Plasmids are also available from Santa Cruz Biotechnology (RFRP CRISPR/Cas9 KO Plasmid, sc-406767 and sc-425634).

Small interfering nucleic acid (siNA) include, for example: microRNA (miRNA), small interfering RNA (siRNA), double-stranded RNA (dsRNA), and short hairpin RNA (shRNA) molecules. An siNA useful in the invention can be unmodified or chemically-modified. Other inhibitor molecules that can be used include ribozymes, peptides, DNAzymes, peptide nucleic acids (PNAs), triple helix forming oligonucleotides, antibodies, and aptamers and modified form(s) thereof directed to sequences in gene(s), RNA transcripts, or proteins.

The dopaminergic pathway, given the reproducible and consistent induction of components of this pathway in the microarray analysis described herein, is a candidate signaling pathway to regulate Npvf gene expression. Hypothalamic PerVN dopamine neurons have been shown to express GPR147 mRNA. Other neuropepetides associated with food intake and thermogenesis, for example NPY and AGRP do not have consistent responses to the variables of our experiment. Thus, in some embodiments, the methods of the invention also involve targeting the dopamine pathway as an adjunct therapy. In some embodiments the dopamine pathway is activated in order to promote energy utilization. The dopamine pathway is well known in the art. One method for activating the dopamine pathway is to administer dopamine to the subject. Other methods are known.

The methods for promoting energy utilization in a subject are useful for treating disorders such as obesity. Treating obesity in a subject means to stabilize, reduce, ameliorate or eliminate a sign or symptom of the obesity in the subject, or to reduce or prevent further development of obesity in the subject. Obesity refers to a subject having a body mass index greater than normal, and typically greater than 30.0 (and thus includes the states of significant obesity, morbid obesity, super obesity, and super morbid obesity and/or to a subject having over 25% or 30% body fat.

The invention in other aspects is a method for treating a wasting disorder in a subject by administering to the subject an effective amount of a Npvf pathway inducing agent to reduce energy utilization. Wasting disorders are disorders in which the body does not have available sufficient energy sources to support energy utilization. In these disorders it is desirable to slow energy utilization. An example of a wasting disorder is anorexia nervosa.

These disorders are treated by administering to the subject an Npvf pathway inducing agent. An Npvf pathway inducing agent as used herein refers to a compound that promotes the activity or expression of a component of the Npvf pathway. Components of the Npvf pathway include but are not limited to Npvf, RFRP and NPFF-R. Npvf pathway inducing agents include but are not limited to exogenous Npvf, including Npvf expression vectors and Npvf mRNA, exogenous RFRP peptides and Npvf activators including NPFF-R1 agonists and NPFF-R2 agonists.

The methods of the invention are useful for treating a subject in need thereof. A subject in need thereof can be a subject who has obesity or a wasting disorder. For example, a subject in need thereof can be a patient who is diagnosed with obesity or a wasting disorder.

In its broadest sense, the terms “treatment” or “to treat” refer to both therapeutic and prophylactic treatments. If the subject in need of treatment is experiencing a condition (i.e., has or is having a particular condition), then “treating the condition” refers to ameliorating, reducing or eliminating one or more symptoms associated with the disorder or the severity of the disease or preventing any further progression of the disease. If the subject in need of treatment is one who is at risk of having a condition, then treating the subject refers to reducing the risk of the subject having the condition or preventing the subject from developing the condition.

A subject shall mean a human or vertebrate animal or mammal including but not limited to a dog, cat, horse, cow, pig, sheep, goat, turkey, chicken, and primate, e.g., monkey.

Therapeutic compounds associated with the invention may be directly administered to the subject or may be administered in conjunction with a delivery device or vehicle. Delivery vehicles or delivery devices for delivering therapeutic compounds to surfaces have been described. The therapeutic compounds of the invention may be administered alone (e.g., in saline or buffer) or using any delivery vehicles known in the art.

The term effective amount of a therapeutic compound of the invention refers to the amount necessary or sufficient to realize a desired biologic effect. For example, an effective amount of a therapeutic compound associated with the invention may be that amount sufficient to ameliorate one or more symptoms of obesity in an obese subject. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular therapeutic compounds being administered, the size of the subject, or the severity of the disease or condition. One of ordinary skill in the art can empirically determine the effective amount of a particular therapeutic compound associated with the invention without necessitating undue experimentation.

Subject doses of the compounds described herein for delivery typically range from about 0.1 μg to 10 mg per administration, which depending on the application could be given daily, weekly, or monthly and any other amount of time there between. The doses for these purposes may range from about 10 μg to 5 mg per administration, and most typically from about 100 μg to 1 mg, with 2-4 administrations being spaced days or weeks apart. In some embodiments, however, parenteral doses for these purposes may be used in a range of 5 to 10,000 times higher than the typical doses described above.

In some embodiments a compound of the invention is administered at a dosage of between about 1 and 10 mg/kg of body weight of the mammal. In other embodiments a compound of the invention is administered at a dosage of between about 0.001 and 1 mg/kg of body weight of the mammal. In yet other embodiments a compound of the invention is administered at a dosage of between about 10-100 ng/kg, 100-500 ng/kg, 500 ng/kg-1 mg/kg, or 1-5 mg/kg of body weight of the mammal, or any individual dosage therein.

The formulations of the invention are administered in pharmaceutically acceptable solutions, which may routinely contain pharmaceutically acceptable concentrations of salt, buffering agents, preservatives, compatible carriers, and optionally other therapeutic ingredients.

For use in therapy, an effective amount of the therapeutic compound associated with the invention can be administered to a subject by any mode that delivers the therapeutic agent or compound to the desired surface, e.g., mucosal, systemic. Administering the pharmaceutical composition of the present invention may be accomplished by any means known to the skilled artisan. Preferred routes of administration include but are not limited to oral, parenteral, intramuscular, intranasal, sublingual, intratracheal, inhalation, ocular, vaginal, rectal and intracerebroventricular.

For oral administration, the therapeutic compounds of the invention can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross linked polyvinyl pyrrolidone, agar, or alginic acid or a salt thereof such as sodium alginate. Optionally the oral formulations may also be formulated in saline or buffers, i.e., EDTA for neutralizing internal acid conditions or may be administered without any carriers.

Also specifically contemplated are oral dosage forms of the above component or components. The component or components may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Other polymers that could be used are poly-1,3-dioxolane and poly-1,3,6-tioxocane. Preferred for pharmaceutical usage, as indicated above, are polyethylene glycol moieties.

The location of release may be the stomach, the small intestine (the duodenum, the jejunum, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the therapeutic agent or by release of the biologically active material beyond the stomach environment, such as in the intestine.

To ensure full gastric resistance a coating impermeable to at least pH 5.0 is preferred. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This can include sugar coatings, or coatings which make the tablet easier to swallow. Capsules may consist of a hard shell (such as gelatin) for delivery of a dry therapeutic i.e., powder; for liquid forms, a soft gelatin shell may be used. The shell material of cachets could be thick starch or other edible paper. For pills, lozenges, molded tablets or tablet triturates, moist massing techniques can be used.

The therapeutic can be included in the formulation as fine multi particulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the therapeutic agent may be formulated (such as by liposome or microsphere encapsulation) and then further contained within an edible product, such as a refrigerated beverage containing colorants and flavoring agents.

One may dilute or increase the volume of the therapeutic with an inert material. These diluents could include carbohydrates, especially mannitol, a lactose, anhydrous lactose, cellulose, sucrose, modified dextrans and starch. Certain inorganic salts may be also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrates include but are not limited to starch, including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Other forms of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An anti frictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the therapeutic into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. The list of potential non ionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the therapeutic agent either alone or as a mixture in different ratios.

Pharmaceutical preparations which can be used orally include push fit capsules made of gelatin, as well as soft, sealed capsules made of gelatin and a plasticizer, such as glycerol or sorbitol. The push fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such micro spheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

Also contemplated herein is pulmonary delivery of the therapeutic compounds of the invention. The therapeutic agent is delivered to the lungs of a mammal while inhaling and traverses across the lung epithelial lining to the blood stream. Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art. Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Colo.; the Ventolin metered dose inhaler, manufactured by Glaxo Inc., Research Triangle Park, North Carolina; and the Spinhaler powder inhaler, manufactured by Fisons Corp., Bedford, Mass.

All such devices require the use of formulations suitable for the dispensing of therapeutic agent. Typically, each formulation is specific to the type of device employed and may involve the use of an appropriate propellant material, in addition to the usual diluents, and/or carriers useful in therapy. Also, the use of liposomes, microcapsules or microspheres, inclusion complexes, or other types of carriers is contemplated. Chemically modified therapeutic agent may also be prepared in different formulations depending on the type of chemical modification or the type of device employed.

Formulations suitable for use with a nebulizer, either jet or ultrasonic, will typically comprise therapeutic agent dissolved in water at a concentration of about 0.1 to 25 mg of biologically active compound per mL of solution. The formulation may also include a buffer and a simple sugar (e.g., for stabilization and regulation of osmotic pressure). The nebulizer formulation may also contain a surfactant, to reduce or prevent surface induced aggregation of the compound caused by atomization of the solution in forming the aerosol.

Formulations for use with a metered dose inhaler device will generally comprise a finely divided powder containing the therapeutic agent suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2 tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing therapeutic agent and may also include a bulking agent, such as lactose, sorbitol, sucrose, or mannitol in amounts which facilitate dispersal of the powder from the device, e.g., 50 to 90% by weight of the formulation. The therapeutic agent should most advantageously be prepared in particulate form with an average particle size of less than 10 mm (or microns), most preferably 0.5 to 5 mm, for most effective delivery to the distal lung.

Intra-nasal delivery of a pharmaceutical composition of the present invention is also contemplated. Intra-nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextran.

For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

Alternatively, a plastic squeeze bottle with an aperture or opening dimensioned to aerosolize an aerosol formulation by forming a spray when squeezed is used. The opening is usually found in the top of the bottle, and the top is generally tapered to partially fit in the nasal passages for efficient administration of the aerosol formulation. Preferably, the nasal inhaler will provide a metered amount of the aerosol formulation, for administration of a measured dose of the drug.

The agents, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The therapeutic compounds of the invention and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the invention contain an effective amount of a therapeutic compound of the invention optionally included in a pharmaceutically-acceptable carrier. The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

The therapeutic agents of the invention may be delivered with other therapeutics for treating obesity.

### EXAMPLE

In order that the invention described herein may be more fully understood, the following example is set forth. The example described in this application is offered to illustrate the compounds, pharmaceutical compositions, and methods provided herein and are not to be construed in any way as limiting their scope.

### Example 1

### Materials and Methods

### Animals

Breeding pairs of C57BL/6J.+/+, C57BL/6J.Ucp1+/− and C57BL/6J.Lep+/− mice were obtained through the generosity of Dr. Martin Klingenspor of the Technical University of Munich, Germany. All procedures concerned with breeding, housing, maintenance and experimental treatment of the mice were approved by the Local Animal Care and Use Committee for University of Warmia and Mazury, Olsztyn.

### Metabolic and Molecular Assays

Mice were anesthetized by the solution of ketamine, xylopan and chlorpromazine (26.6 mg/ml, 1.67 mg/ml and 0.53 mg/ml, respectively, 40 μl/10 g body weight) and the blood was collected through heart puncture to EDTA coated tubes. After decapitation, interscapular brown adipose tissue depot (iBAT), inguinal white adipose tissue depot (iWAT), and hypothalamus were removed, rapidly frozen in liquid nitrogen and stored at −80° C. for subsequent preparation of total RNA. The blood was centrifuged for 10 min at 3,000 g, 4° C. Plasma was removed and stored at −80° C. until assayed.

### Plasma Hormones and Metabolites

Plasma insulin and leptin were measured by enzyme-linked immunosorbant assay with commercial kits (Wide range mouse insulin immunoassay kit, Biorbyt Ltd., Cambridge, UK; Mouse/rat leptin ELISA kit, Phoenix Pharmaceuticals, Inc., Burlingame, Calif., United States, respectively). Assessment of FFA in plasma was performed with plasma non-esterified free fatty acid detection kit (Zenbio, Inc., Research Triangle Park, NC, United States).

### Quantitative Real-Time PCR

Total RNA was isolated from adipose tissue, liver and hypothalamus using TRI Reagent and BCP phase separation reagent (Molecular Research Center Inc. Cincinnati, Ohio, United States). RNA was further purified by using the RNAeasy minikit (QIAGEN, Valencia, Calif., United States) and stored at −80° C. in RNase-free H₂O with addition of SUPERase-In (Ambion, Austin, Tex., United States) for RNase protection. Quality and quantity of RNA was determined using UV spectrophotometry (Nanodrop) and agarose gel visualization of intact RNA. Quantitative RT-PCR using TaqMan probes and primers (Applied Biosystems, Foster City, Calif., United States) was performed with standard curves generated using pooled RNA isolated from corresponding tissues collected from eight 8 week old C57BL/6J.+/+ mice. Probe and primer sequences used to perform the analyses are available upon request. All the gene expression data were normalized to the level of cyclophilin b.

### Microarray Analysis of Gene Expression in the Hypothalamus of Lep−/− and Lep+/+Mice

Total RNA was isolated from the hypothalamus of 8 Lep−/− and 8 Lep+/+ mice maintained at 24 and 6° C., as described above. RNA with RNA Integrity number higher than 8.5 (Agilent 2100 Bioanalyzer, Agilent Technologies, Santa Clara, Calif.) was used for microarray analysis of each individual mouse. RNA was amplified, labeled and hybridized onto chips containing over 56,000 probes of mouse genes (Agilent Single Color SurePrint G3 Mouse GE 8x60K Microarray Kit, G4852A, Agilent Technologies) according to manufacturer's guidelines. Agilent Feature Extraction software was used for array image analysis. Absolute and comparative analyses were performed using the GeneSpring GX 10 (Agilent Technologies). Quality control filtering after quantile normalization resulted in approximately 33,000 probes. Probes that were not above microarray background signal or whose sequences could not be mapped to Ensembl transcripts were discarded. Genes were considered significantly down-regulated or up-regulated if the fold-change was less than −1.4 or greater than 1.4, respectively, and the FDR-corrected P-value was less than 0.05. To validate the reliability of the results obtained from the microarray analysis, we performed qRT-PCR for all genes of interest.

### Statistical Analysis

Graphs were created with the GraphPad Prism Software (Version 6.0, GraphPad Software, Inc.; La Jolla, USA). All data sets were analyzed using Student's test for groups (GraphPad Prism Software). Data are presented as means±SEM. Differences between the means for all tests were considered statistically significant if P<0.05.

Energy Expenditure During Cold Exposure of Mice with DIO

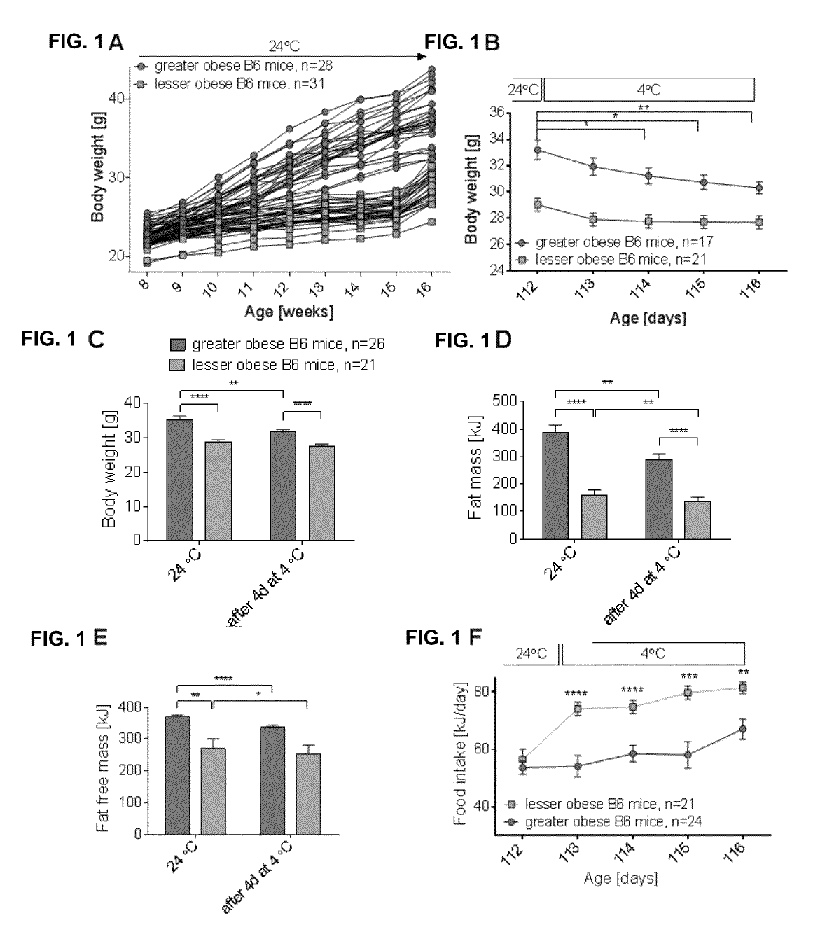

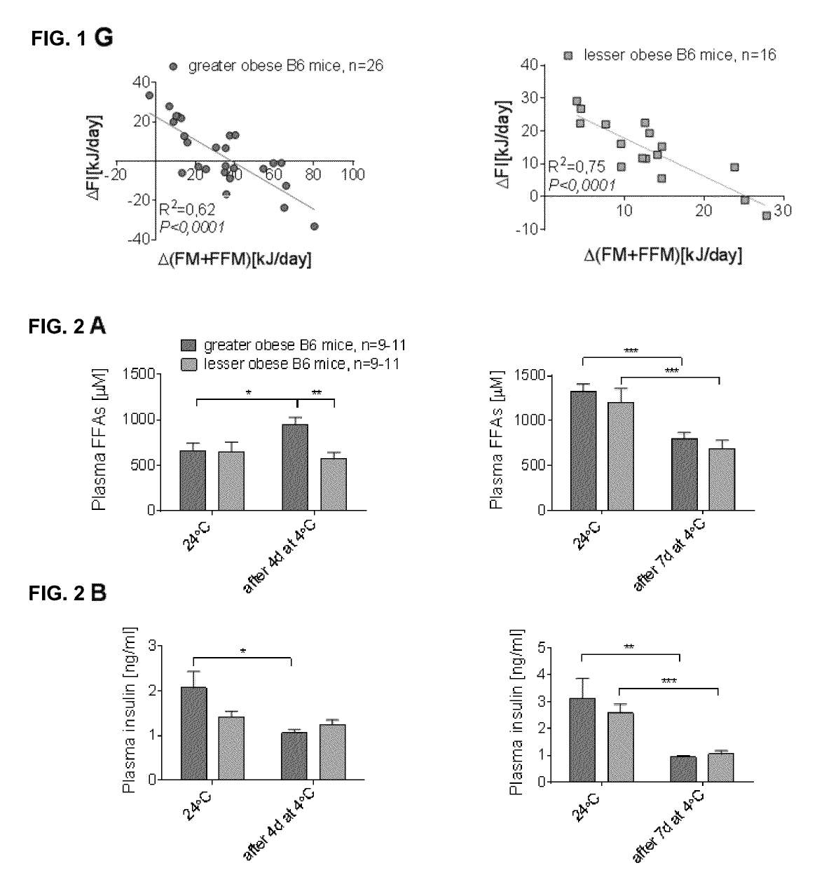

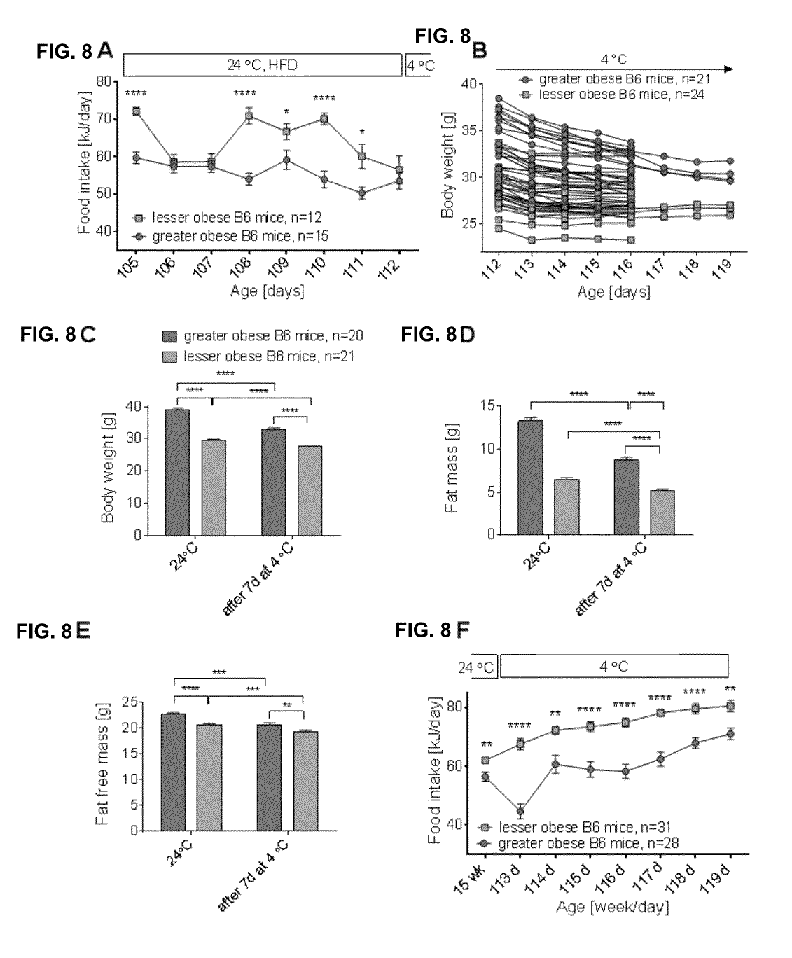

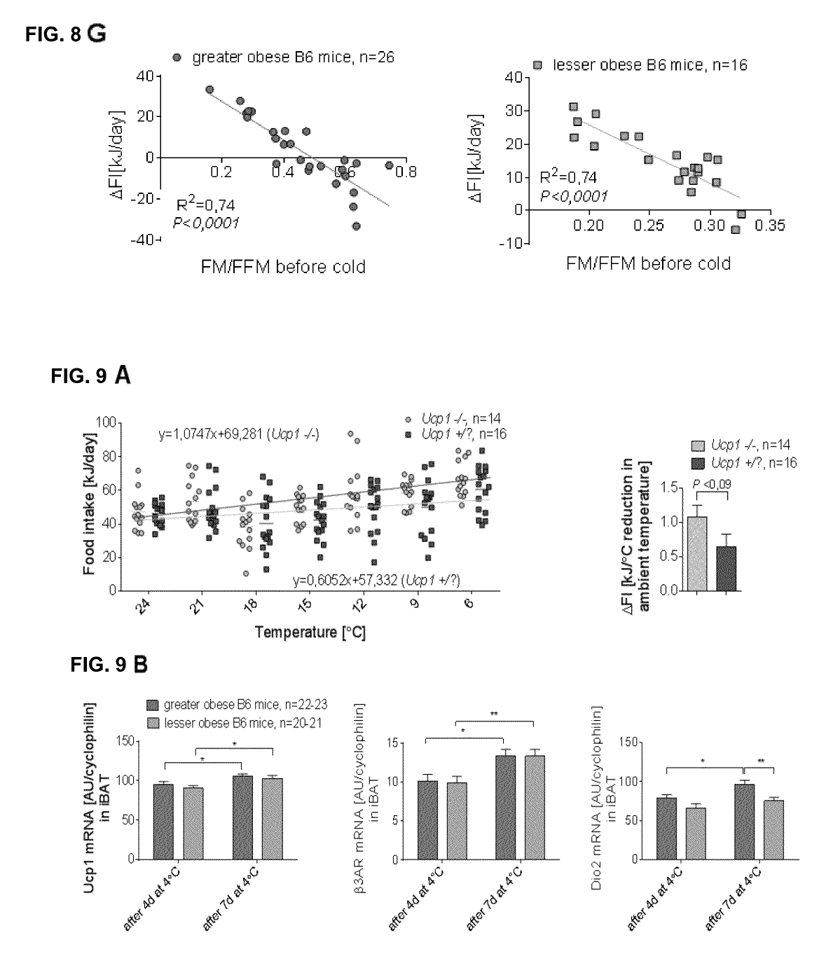

The goal of this protocol was to generate a series of mice with a range of adiposities by use of a high fat diet to determine the effects of cold exposure on changes in food intake and endogenous energy stores. Between 3 and 8 weeks of age male B6 mice, fed a low fat diet, were monitored by NMR to identify 2 groups of mice that would form the lesser and greater obese groups. The greater obese group was fed a high-fat diet (AIN-76A with 33% hydrogenated coconut oil, 58 kcal % fat) from the 8^{th} to 16th week to establish a range of mice with a higher adiposity index. The lesser obese would continue to be fed the low fat diet (PicoLab Rodent Diet 20, LabDiet 5053, 11.9 kcal % fat) for 7 weeks and then the high fat diet for the 16^{th} week. Mice with body weights and fat mass ranging from 24.4 to 43.8 g and 3.7 to 18.8 g, respectively, were formed (FIGS. 1A-D). After the 8^{th} week all mice were individually housed. In addition to having mice with a variation in adiposity, food intake of the lesser and greater obese mice was measured for the 16^{th} week, just prior to being exposed to the cold. Food intake, initially varied in the lesser obese mice when presented with a highly palatable high-fat diet for the first time; however, on the last day before cold exposure, there was no significant difference in food intake between the lesser and greater obese mice (FIG. 8A).

Breeding pairs of C57BL/6J+/+ mice were housed at standard temperature (24±1° C.) and maintained in ventilated rooms under a standard-day photoperiod (12:12-h light-dark period, lights on from 0700 to 1900 h) with free access to food (PicoLab Rodent Diet 20, LabDiet 5053, 11.9 kcal % fat) and water. At 21 days of age male progeny were weaned and housed in groups of 3-5 in plastic cages with fresh sawdust bedding. Body weight and body composition by NMR (Bruker, BioSpin, Germany) were monitored until mice were 8 weeks of age, at which time mice were divided into two nutritional groups matched for similar mean body mass and body fat content to form the lesser and greater obese groups. By 8 weeks of age, when mice were still on a low fat diet, their body weights ranged from 19.2-25.5 g (FIG. 1A). Based on the NMR analysis, the distribution in body weights was mainly caused by differences in fat mass, with only a small contribution from fat free mass. At this time point mice with higher endogenous fat stores were fed a high-fat diet (AIN-76A) for 8 week to produce the cohort of greater obese mice. Correspondingly, also at 8 week of age, leaner mice were fed standard chow (LabDiet 5053) for an additional 7 weeks and high-fat diet for 1 week (the 16^{th} week) to form the lesser obese group. The aim of such a dietary intervention was to establish as broad a range in adiposity as possible between two groups of mice and at the same time to induce metabolic adaptations associated with high-fat feeding in the lesser obese mice at the time of cold exposure.

In order to determine the relative contribution of food intake and endogenous energy reserves to fuel cold-induced thermogenesis, individually housed mice in the lesser and greater obese groups of mice were transferred to a cold room at 4° C. for either 4 or 7 days. Food intake (high fat; AIN-76A) and body weight were measured daily and body composition was analyzed by NMR at the end of the cold exposure.

To observe the effects of cold-induced hyperphagia on DIO mice that were returned to an ambient temperature of 24° C., adult C57BL6/J+/+ mice were fed a high-fat diet (AIN-76A) from 8 to 16 weeks of age then transferred to 4° C. until food intake stabilized over a course of 16 days. Mice were returned to an ambient temperature of 24° C. and the suppression of food intake was monitored for an additional month.

### The Effects of Leptin Deficiency on the Relative Utilization of Food Intake and Endogenous Energy Stores During Cold-Induced Energy Expenditure.

Eight week-old wild-type C57BL/6J.Lep+/+, (n=13), heterozygous C57BL/6J.Lep+/−, (n=10) and homozygous null C57BL/6J.Lep−/−, (n=19) mice of both sexes were individually housed in an environmentally controlled chamber (EHRET GmbH, Emmendingen, Germany) and fed standard rodent chow pellets (LabDiet 5053, 11.9 kcal % fat) during the whole experiment. Housing conditions (photoperiod, air changes) were the same as in experiment 1. After 3 days of habituation to the chamber environment (24° C.), food intake at 24° C. was recorded during 3 consecutive days using high-precision food weighting sensors (PhenoMaster System, TSE Systems GmbH, Bad Homburg, Germany), then, the temperature in the chamber was reduced by 3° C./day to 6° C. at which time mice were kept at 6° C. for additional 2 days. Since Lep−/− mice cannot tolerate an acute reduction in ambient temperature to 6° C., this gradual reduction in ambient temperature was implemented to adapt the mice to the cold (20, 21). Body composition was analyzed by NMR before and after the cold test.

The Effects of UCP1 Deficiency on Cold-Induced Energy Expenditure.

Both backcross (C57BL/6J.Ucp1−/−×C57BL/6J.Ucp1+/−) and intercross (C57BL/6J.Ucp1+/−×C57BL/6J.Ucp1+/−) matings were used to generate C57BL/6J.Ucp1−/− mice together with heterozygous and homozygous normal (wild-type) controls (23). Male mice were fed chow diet (LabDiet 5053, 11.9 kcal % fat) until 8 weeks of age. Obesity was induced in mice by feeding them a high-fat diet (AIN-76A, 58 kcal % fat) from 8 to 16 weeks of age. At 16 weeks of age mice were transferred to the temperature-controlled chamber (EHRET GmbH). Housing conditions, the protocol for exposing the mice to the cold and phenotyping of adiposity and food intake was the same as that described for part (a).

Regulation of Npvf Expression in Mouse Hypothalamus Under Variable Thermogenic Conditions

To establish the influence of different ambient temperature on Npvf expression mice were kept in climate-controlled rodent incubators. To evaluate the effects of the β adrenergic receptor agonist on Ucp1 and Npvf, thermoneutrally acclimated mice were injected subcutaneously with 1 mg kg⁻¹ CL 316,246 or saline for 7 days.

All mice used in the following studies were adult wild-type C57BL/6J+/+ mice. Mice were placed in individual cages with free access to food (LabDiet 5053) and water. At the end of each experiment mice were sacrificed and hypothalamus was dissected in order to measure the level of Npvf mRNA expression. To establish the influence of different ambient temperature on Npvf expression mice were kept in climate-controlled rodent incubators set to 29° C. and 17° C. for the period of 2 weeks prior to sacrifice. Additional experiment was performed to observe the kinetics of changes in the level of Npvf mRNA in the cold. All mice used in this study were first allowed to acclimate to 29° C. for 2 weeks before cold challenge. Temperature of the housing unit was then transitioned from 29° C. to 6° C. and mice were cold-challenged for 6 h, 12 h or 24 h.

### Results

Energy Expenditure During Cold Exposure of Mice with DIO

The high natural variation in susceptibility to DIO among genetically identical B6 mice fed a high-fat diet (HFD) generated a cohort of mice in which body weight ranged between 24.4 and 43.8 g (FIG. 1A) (18). Reducing the ambient temperature from 24 to 4° C. resulted in an immediate lowering in body weight that was highest on day one and gradually diminished during the succeeding days (FIG. 1B, FIG. 8B). Although lesser and greater (range of body weight) obese mice showed the same response, the absolute weight loss was larger in the greater obese group than the lesser obese group (FIGS. 1B-1C, FIGS. 8B-8C). Fat mass was the major endogenous substrate fueling thermogenesis (FIG. 1D and FIG. 8D). In the greater obese group, after 4 days at 4° C. 97.5 kJ of energy came from fat mass and 33.8 kJ from fat free mass. For the lesser obese group, 30 kJ came from fat mass and 22.9 kJ from fat-free mass (FIGS. 1D-1E). After one day at 4° C. both groups of mice experienced a slight decline in body temperature (1-2° C.), however, by the 2^{nd} day at 4 C all mice were able to thermoregulate and maintain their body temperature at the level at which they started (36±1° C.).

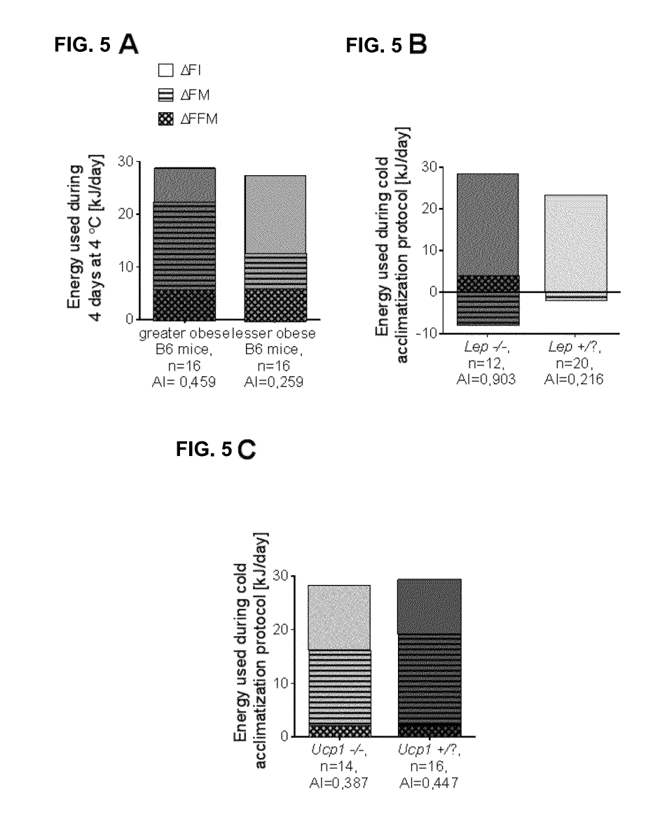

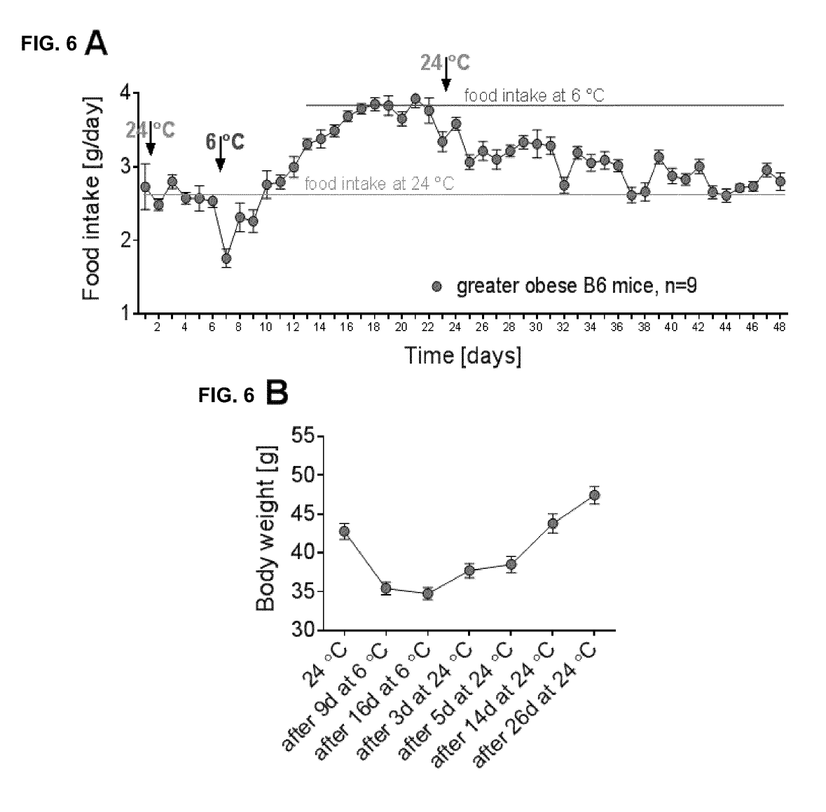

We set out to determine next where the energy for thermogenesis was coming from since the lesser obese group utilized less of their endogenous energy reserves during cold exposure than the greater obese. For this we measured food intake. After 16 weeks on the dietary regime at 24° C., as described in the Methods, food intake was 56.5 kJ/day for the lesser obese and 53.6 kJ/day for the greater obese (FIG. 1F). When mice were transferred to 4° C., food intake immediately increased in the lesser obese mice to 75 kJ/day (35% increase) and to 84 kJ/day (50% increase) after 1 and 4 days, respectively; the increase in food intake was smaller in the greater obese mice going to 55 kJ/day (2% increase) and 67 kJ/day (25% increase), respectively, after 1 and 4 days in the cold. With increasing time at 4° C. the difference in food intake between the lesser and greater obese groups was reduced (FIG. 8F), consistent with the diminishing difference in fat mass. After the first day of cold exposure the difference in food consumption between mice from 2 cohorts equaled 20.06±4.53 kJ, after 4 days at 4° C. it was 14.44±4.23 kJ (FIG. 1F) and only 9.53±2.87 kJ after 7 days at 4° C. (FIG. 8F). Cold-induced thermogenesis is associated with increased consumption of fuel reserves and, as evident in FIG. 1F and FIG. 8F, mice with lower endogenous fuel reserves compensate by increasing food intake, a process that apparently increases with time as endogenous fuel reserves become depleted. After 4 days at 4° C., regardless of the level of obesity present in the animals before cold exposure, cumulative energy coming from feeding and mobilized endogenous energy stores was comparable in the greater and lesser obese mice (FIG. 5A).

For both lesser and greater obese animals linear regression analysis revealed a strong negative correlation between energy reserves (fat and fat free mass) mobilized per day and daily energy consumed during time spent in the cold (R²=0.62 for greater obese mice and R²=0.75 for lesser obese after 4 days in the cold) (FIG. 1G). An equally strong negative relationship was observed when values of adiposity index calculated for each mouse before cold exposure were plotted against daily food intake during 4 days at 4° C. (R²=0.74 for both greater and lesser obese mice) (FIG. 8G). These findings suggest a strategy by which energy requirements for survival in the cold are adjusted by a mechanism that depends on the body composition of the mouse; mice with small fat stores preferentially increase energy intake and conserve endogenous energy reserves, whereas mice with abundant fat stores primarily utilize these reserves when energy is in demand (FIG. 5A).

### Leptin Resistance and the Cold Challenge

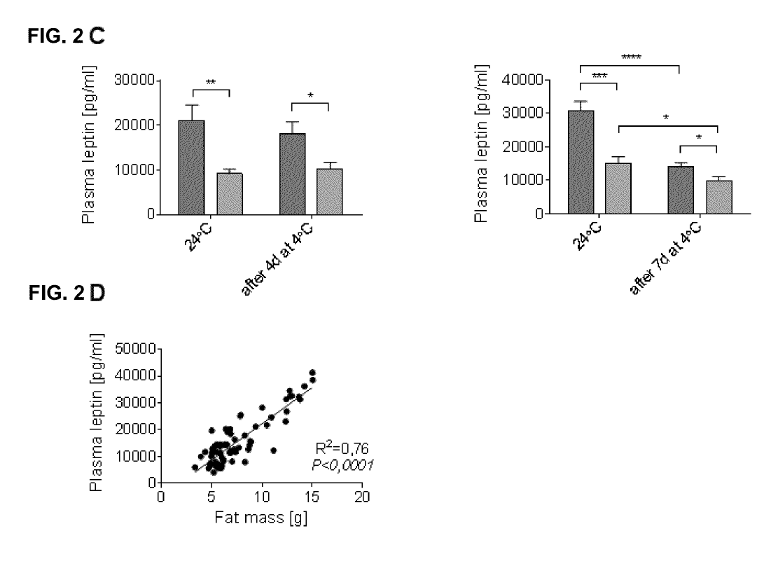

At 24° C. there were no differences in the level of plasma free fatty acids (FFAs) and insulin between the groups (FIG. 2A-B). After 4 days at 4° C., greater obese mice had significantly elevated levels of circulating FFAs in comparison to lesser obese, consistent with increased fat mobilization in the greater obese animals. Substantial fat mass loss after 7 days of cold exposure resulted in reduced plasma FFAs in both groups of mice. Similarly, after 7 days at 4° C., circulating insulin was decreased in greater and lesser obese mice compared to 24° C. (FIG. 2B). Leptin levels did not drop during the first 4 days at 4° C., only after 7 days in the cold did highly significant reductions in leptin levels occur (FIG. 2C).

At 24° C. leptin levels were positively correlated with adiposity (FIG. 2D) indicating that leptin resistance should be higher in the greater obese mice than in the lesser obese, predicting a defense of adipose stores and higher food intake in greater obese mice. However, from the very beginning of cold exposure a defense of the fat status in mice with leptin levels predictive of leptin resistant was not observed. In fact the opposite was observed, with food intake reduced and fat utilization increased in the greater obese mice compared to the lesser obese. Since this phenotype of DIO mice with leptin resistance in the cold was not predicted, we evaluated the response of Lep−/− mice to a cold challenge.

### The Effects of Leptin Deficiency on the Relative Utilization of Food Intake and Endogenous Energy Stores During Cold-Induced Energy Expenditure

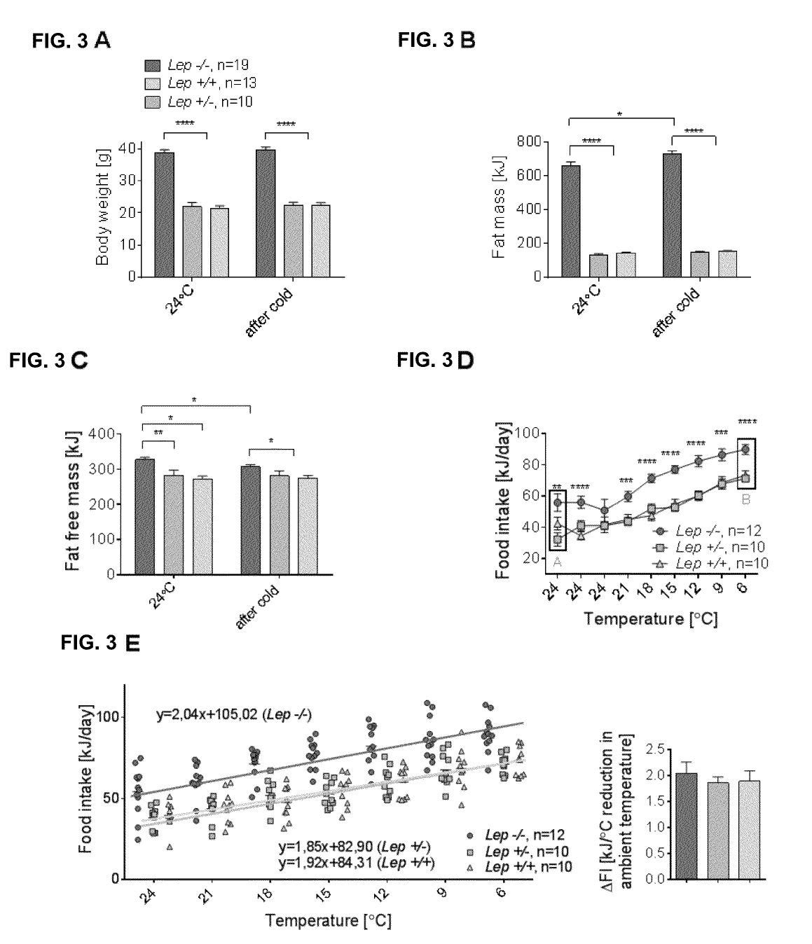

Mice deficient in either leptin or the leptin receptor are cold intolerant when acutely exposed to 4° C.; however, they are able to adapt to a lower temperature if the exposure is gradual (19-21), thereby enabling an analysis of energy utilization during a cold challenge. Although there were large differences in body mass and composition between Lep+/? and Lep−/− mice fed a low fat chow diet at 24° C., after 9 days in the cold, neither genotype showed significant changes in body weight mass or composition (FIGS. 3A-3C). With no reduction in endogenous energy reserves we looked to an increase in food intake. At 24° C. average daily food intake was about 40% higher in leptin-deficient than in the Lep+/? control mice, as previously observed by Coleman (20) (FIG. 3D). One would anticipate that this source of energy would be used to fuel thermogenesis, however, reducing the ambient temperature by 3° C. per day resulted in an increase in food intake in both control and Lep−/− mice. This food intake curve is displaced upward by an amount corresponding to the difference in food intake between control Lep+/? and Lep−/− mice at 24° C. (FIG. 3D). Therefore, the rate of increase in food intake per degree Celsius reduction in ambient temperature by the control mice and Lep−/− mice was essentially indistinguishable (FIG. 3E). The most striking observation was that Lep−/− mice, already hyperphagic at 24° C., further increased their food intake under a cold challenge. They sensed that existing fat stores were unavailable and compensated by increasing food intake in a leptin-independent manner. This feeding behavior in the cold underscores the inability of mice with leptin-deficiency to utilize endogenous fat reserves; furthermore it also shows that in the face of a cold challenge fuel for thermogenesis must come from food intake. After correcting for the slight changes in body composition that occurred in mice upon cold exposure, the total energy used for cold-induced thermogenesis was equal in leptin-deficient and control mice (FIG. 5B). This homogeneity in metabolic phenotypes for normal and leptin-deficient mice suggests that the energy required for thermogenesis under these conditions is acquired by increasing food intake through a leptin-independent mechanism.

### The Effects of UCP1 Deficiency on Cold-Induced Energy Expenditure

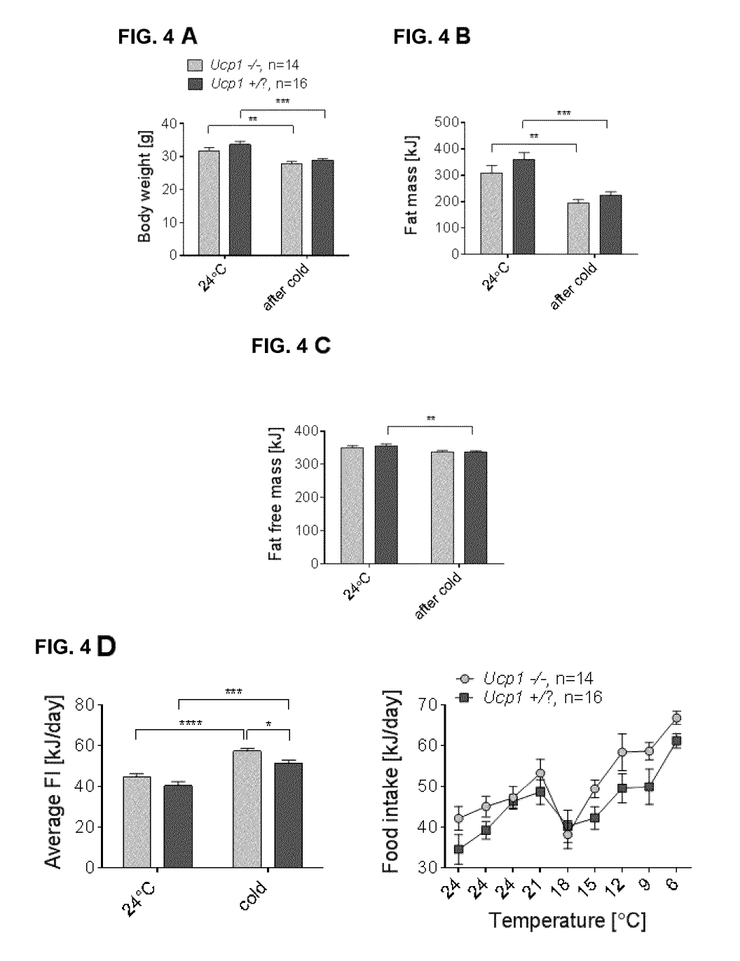

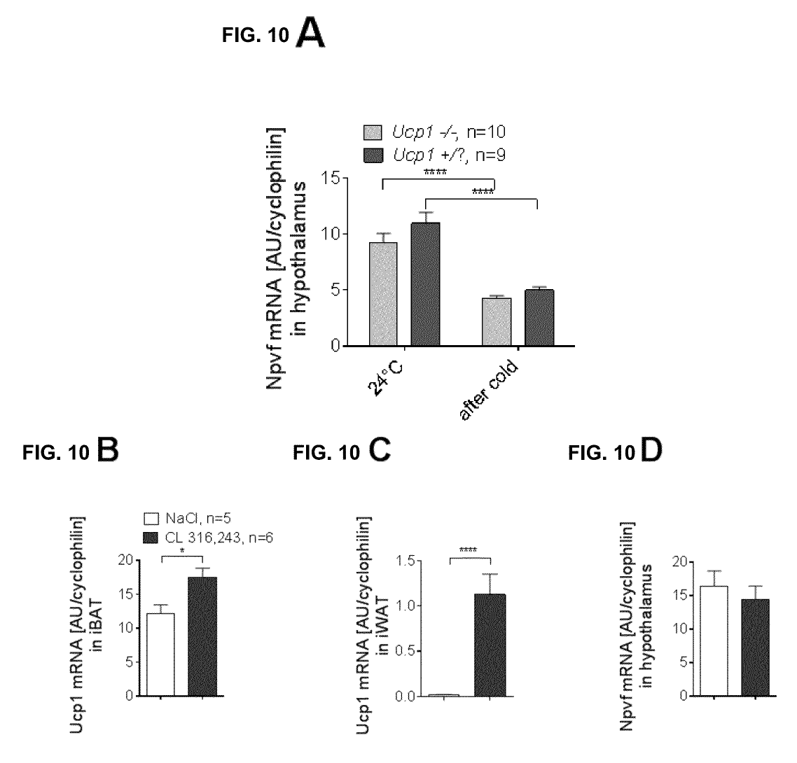

It is assumed that non-shivering thermogenesis of brown fat is essential for providing the heat to protect the animal from the cold. Indeed Ucp1−/− newborn mice on either the B6 and 129 genetic backgrounds cannot survive the first days of birth in a breeding room maintained at ˜23° C. and Ucp1−/− adult mice acutely exposed to the cold at 4° C. will succumb within 5 hours (22, 23). However, similar to Lep−/− mice, Ucp1−/− mice can adapt to the cold (24). Ucp1−/− and Ucp1+/? mice were exposed to the cold using the same protocol as that used for Lep−/− mice, except that DIO was first induced at 24° C. as with the greater and lesser obese mice (FIG. 1A). The level of obesity for the Ucp1+/? resembled that of the greater obese B6.+1+ mice, whereas the Ucp1−/− mice resembled the lesser obese mice (FIGS. 4A-4C), even though they were fed the HFD for the full 8 weeks. This is expected, since at 24° C. Ucp1−/− mice are resistant to DIO (23). At 24° C. food intake was similar for mutant and control mice, whereas the daily energy intake during cold adaptation was higher for Ucp1−/− mice (FIG. 4D, FIG. 9A). Similar to the results of the initial experiment wild type B6 mice, Ucp1+/? mice with the greater obese phenotype preferentially lost fat mass during cold adaptation, whereas the Ucp1−/− mice with the lesser obese phenotype preferentially increased food intake (FIG. 5C). Thus, energy balance and substrate utilization in DIO Ucp1−/− mice during cold exposure resembles that of lesser obese wild-type mice. An important observation is that mice with robust DIO after 8 weeks on a high fat diet at 24° C. will concurrently increase food intake and reduce body weight when transferred to an ambient temperature of 6° C. (FIGS. 6A-6B). They will stabilize both body weight and food intake to a new state of energy balance to maintain body temperature, until they are returned to 24° C. when they return to the level of food intake observed before the cold exposure and resume the characteristic increase in adiposity. It was previously observed the same response of mice fed a high fat diet when energy balance was interrupted with food restriction (18). Accordingly, mice do not assume increased levels of food intake transiently acquired when they are in the cold, rather food intake is set by the requirements for heat production.

In summary, UCP1-dependent brown fat thermogenesis is not required to derive the weight reducing benefits of adapting to the cold and there is no mechanism associated with thermogenesis that will increase food intake of the greater obese to preserve the obese state. There is a mechanism, however, to preserve a minimal adiposity index typified by young adult C57BL/6J mice fed a low fat chow diet. In this experiment with 3 different obesity models estimates of energy expenditure during cold exposure could have been measured by indirect calorimetry; however, the need for appropriate adaptation makes indirect calorimetry impractical and the slight differences, which may be observed, were not critical to the goals of the study. Total energy consumption as shown by 6 experimental groups (FIGS. 5A-5C) indicates that energy expenditure during cold exposure is generally similar for the six groups, with exception that Ucp1−/− mice are metabolically inefficient and have higher O₂ consumption per mouse (26). The difference among groups describes the source of energy for the induction of thermogenesis, endogenous reserves vs food intake, and it is this difference which is the focus of this study.

A Molecular Pathway Associated with Cold Activated Thermogenesis

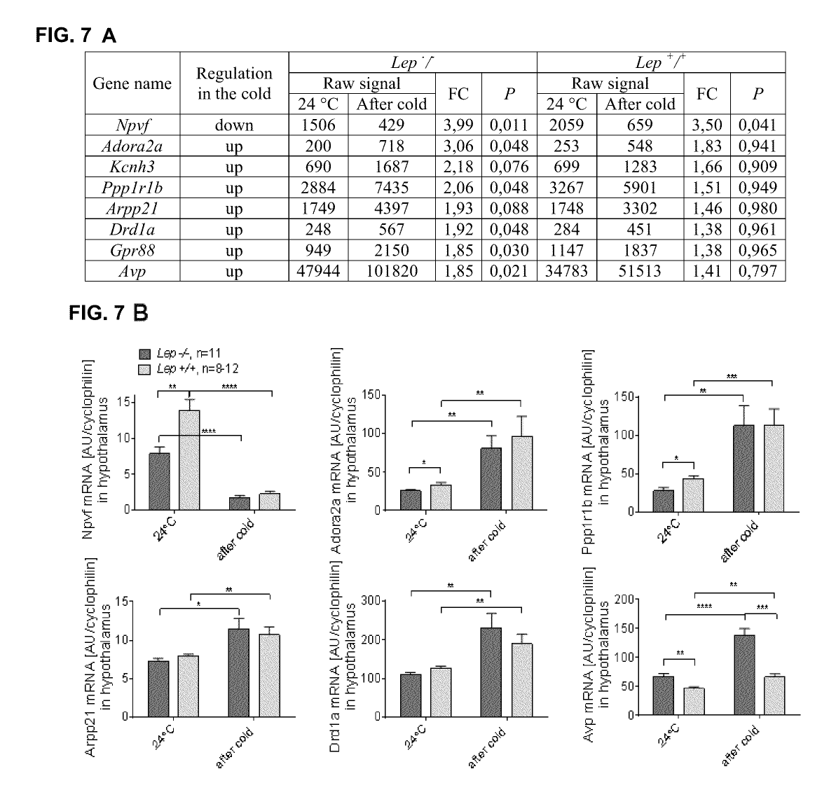

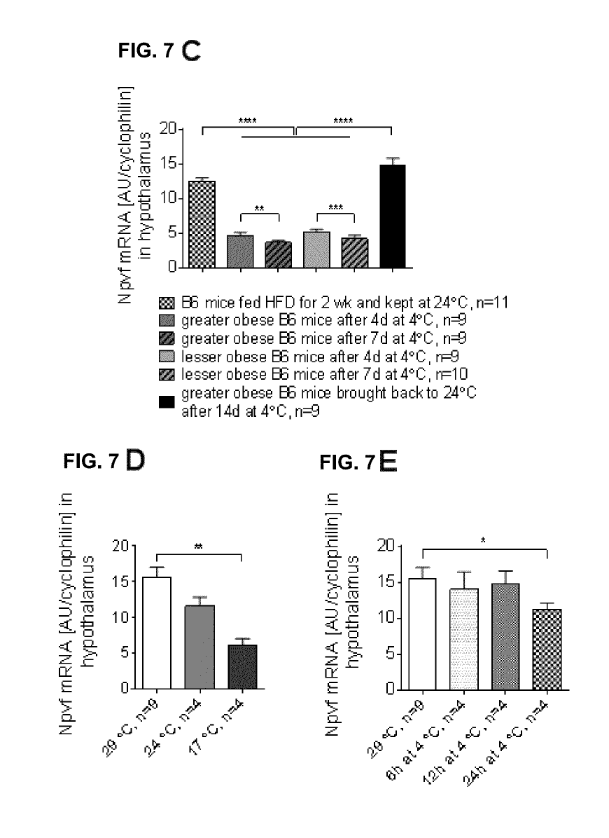

At 24° C. Lep−/− mice are hyperphagic compared to the Lep+/+ or Lep+/− mice (FIG. 3D). Reducing the ambient temperature from 24 to 6° C. was accompanied by a graded parallel increase in food intake, corresponding to approximately 100 kJ of energy for both control and mutant mice (FIG. 3D). Consequently, the same leptin-independent increase in food intake was observed during the transition from 24 to 6° C. in both Lep+/+ and Lep−/−. Since the energy content of Lep+/+ and Lep−/− mice was unchanged during cold exposure, thermogenesis is fueled solely by food intake. Accordingly, we predicted that the same changes in gene expression associated with the central regulation of thermogenesis by the hypothalamus must occur in both Lep+/+ and Lep−/− mice during the transition from 24 to 6° C. Microarray analysis of gene expression was performed on hypothalamic tissue dissected from Lep−/− and Lep+/+ mice kept at different temperature conditions, that is, in mice maintained at 24° C. (point A, FIG. 3D) and in mice in which the ambient temperature had been reduced to 6° C. (point B, FIG. 3D). A small subset of genes in Lep−/− in common with Lep+/+ mice during the transition from 24 to 6° C. were identified (FIG. 7A). Among these genes, neuropeptide VF precursor (Npvf), showed a robust down-regulated expression of 4.0 and 3.5 fold in the hypothalamus of cold-exposed Lep−/− and Lep+/+, respectively. A group of G protein-coupled receptors (GPCRs) including the dopamine receptor DrD1A, adenosine receptor Adora2A, GABA(A) receptor subunit delta GABDR and Gpr88 as well as some of their downstream targets including cAMP-regulated phosphprotein 21 (Arpp21) and protein phosphatase 1 regulatory subunit 1B (Ppp1r1b) were up-regulated 1.4 to 3 fold in both Lep+/+ and −/− mice following cold exposure. Previous studies have implicated some of these GPCRs in addictive behaviors (27, 28). Dopamine, has been implicated as a mechanism in reward-associated feeding behavior (29). Cold exposure also increased the expression of antidiuretic hormone arginine vasopressin Avp gene in both mutant and wild-type animals by 1.8 and 1.4 fold, respectively. Up-regulation of vasopressin mRNA in response to cold was previously observed in rat hypothalamus (30, 31). Each of the genes expressed in parallel in Lep+/+ and −/− mice were validated by qRT-PCR (FIG. 7B).

To further investigate a potential role for Npvf in food intake as a function of cold, its expression in the hypothalamus of mice with different levels of dietary-induced obesity following cold exposure was determined (FIG. 1 and FIG. 8). Similar to the experiment with Lep+/+ and −/− mice, Npvf expression was suppressed in both greater and lesser obese mice after the temperature shift from 24 to 4° C., but its expression was not associated with either adiposity or food intake (FIG. 7C). Lack of association between body energy reserves and hypothalamic Npvf expression was also shown in the recent study in which no significant difference in Npvf mRNA was detected between mice fed high-fat and low-fat diet for 20 weeks (32). In the same study no leptin signaling or co-localization of leptin mRNA in neurons expressing NPVF were detected is mice brain, indicating that a functional interaction between leptin and Npvf is unlikely. Increasing the duration of cold exposure at 4° C. from 1 to 7 days gradually amplifies the reduction in Npvf mRNA levels. Thus the reduction in Npvf expression at lower temperature when a higher level of energy expenditure (EE) is required suggests that Npvf action inhibits EE. Although increased food intake provides for the increase in EE in lesser obese mice, endogenous fat provides the energy in greater obese mice. Since Npvf is similarly suppressed in both lesser and greater obese mice, food intake per se is not the signal determining Npvf expression levels.

Npvf mRNA expression in hypothalamic tissue showed a positive correlation with ambient temperature. Mice kept for 14 days at thermoneutrality (29° C.) had higher expression of Npvf mRNA in hypothalamus than at 24° C. Similarly, 2 weeks at 17° C. resulted in a reduction of mRNA expression to levels below that observed at 24° C. (FIG. 7D). In an independent experiment, DIO mice that were maintained at 4° C. for 14 days and then returned to 24° C. for 25 days restored their levels of Npvf mRNA to that initially observed at 24° C. (FIG. 7C).

Although modulation of Npvf precursor mRNA occurs during cold-stimulated thermogenesis, an involvement of Npvf in the regulation of non-shivering thermogenesis in brown fat is unlikely. Down-regulation of Npvf mRNA was not influenced by the lack of UCP1 protein (FIG. 10A). Acute exposure to the cold requires an immediate response for heat generation and leads to immediate UCP1 production in BAT and WAT. A separate experiment performed to illustrate time-course of changes in the expression of Npvf under low temperature conditions showed that significant suppression in the amount of Npvf mRNA does not occur before 12 h at 4° C.; a significant decrease in the accumulation of Npvf mRNA in hypothalamus is found after 24 h at 4° C. compared to 29° C. (FIG. 7E). Moreover, one week administration of β3-adrenergic agonist CL 316,243 (1 mg/kg of body weight) did not result in the suppression of Npvf mRNA in hypothalamus compared to saline-treated control mice (FIG. 10B-10D), providing evidence that changes in expression of the Npvf gene is not linked to heat production or brown adipocyte induction in peripheral β3-AR-expressing tissue targets.

Expression of CNS and Peripheral Genes Associated with Energy Metabolism

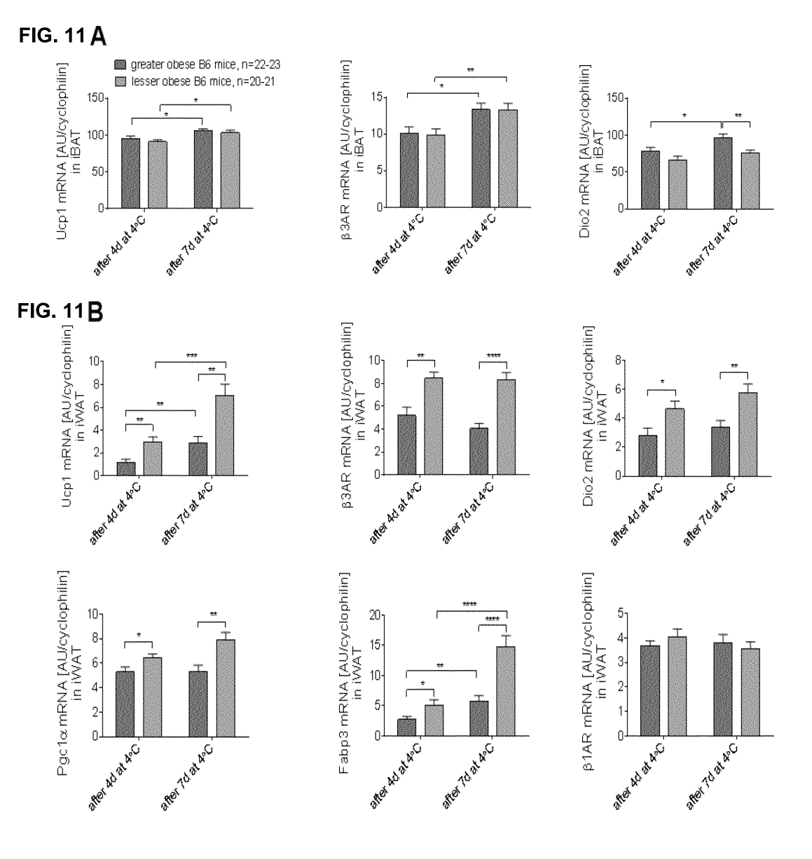

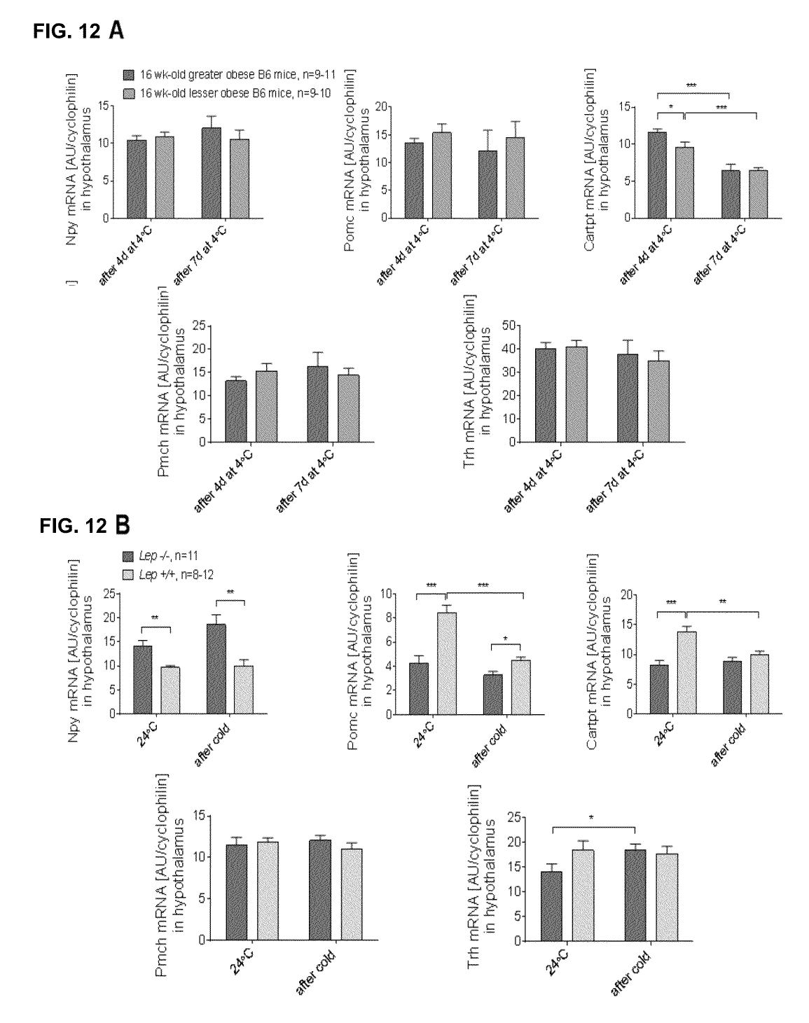

Genes associated with food intake in the hypothalamus and thermogenesis in the adipose tissue were analyzed by qRT-PCR. No patterns in gene expression could illuminate mechanisms associated with the phenotypes described above (see FIG. 11A and FIG. 11B for thermogenic genes in iBAT and iWAT and FIGS. 12A and 12B for neuropepetides of feeding behavior).

### DISCUSSION

We show that the total energy expended by a mouse from food intake and endogenous energy reserves to sustain thermogenesis during cold exposure is independent of the degree of obesity in the animals. This is true in genetically obese Lep−/− mice, chow-fed wild-type mice and in wild-type mice and B6. Ucp1−/− with variable levels of DIO. In chow-fed mice the energy that is necessary to sustain a thermogenic program to maintain body temperature in the cold comes exclusively from feeding, as observed by others (20, 33); however, in a wild-type mouse with diet-induced obesity induced by a high-fat diet, the fuel to support thermogenesis is obtained from endogenous energy reserves (mostly fat) and food intake. In DIO mice the source of energy required to maintain body temperature during cold exposure is determined by the degree of obesity. In DIO mice the energy reserves in fat mass are not privileged or restricted as those in a normal wild-type mouse maintained on a low-fat chow diet, rather they are utilized in proportion to their absolute levels. DIO mice with the highest levels of stored fat immediately mobilize fat, then as these reserves become deleted, food intake becomes progressively a larger contributor to the fuel mix. In contrast, those mice that are at the opposite end of the DIO spectrum, the lesser obese mice, will preferentially increase food intake and use less of their endogenous fuel reserves to support thermogenesis. An important finding is that wild type mice with high levels of adiposity behave in response to cold exposure by the utilization of available energy sources in a manner that is independent of hormonal status, i.e. leptin and insulin.

Interestingly, our observations on body composition-dependent differential fuel selection occurring during cold exposure in DIO mice correlates with findings in exercising human subjects (32). Moderate to intense physical activity performed regularly and on a long-term basis by lean subjects is compensated for by a corresponding change in food intake while body mass is maintained. On the other hand, obese subjects with excess fat storage do not significantly increase food intake and loss of body fat occurs as a consequence (34). A return of mice fed a HFD from 6 to 24° C. leads to a decrease in food intake and increase in adiposity characteristic of their phenotype on a high fat diet (FIGS. 6A-6B). The long-term defense of body weight in humans and mice has been described and discussed as a consequence of under- and over-feeding (35); however, mechanisms associated with a negative energy balance resulting from reduced energy intake during dieting may be different from increased energy expenditure in response to cold-induced energy expenditure, since the latter condition is supported by increased food intake and the neutralization of insulin and leptin resistance (2, 36).

Wild-type B6 (Lep+/− or Lep+/+) mice fed a low fat chow diet exhibited almost no change in endogenous energy reserves, that is, lean mass or fat mass when the ambient temperature was reduced from 24 to 6° C., but they increased food intake. This observation fits with the thermostatic theory proposed by John Brobeck in the late 1940s, which relates the regulation of body temperature to the control of feeding behavior (25). Brobeck summed up his theory by saying: “ . . . animals eat to keep warm and stop eating to prevent hyperthermia”. In the present study, the wild-type B6 mouse maintained energy balance and body composition on a normal diet, when exposed to the cold, by increasing calorie intake. Importantly, the Lep−/− mouse behaved in the same manner, it adapted to the cold by increasing food intake in a manner quantitatively indistinguishable from the normal B6 mouse and it preserved its endogenous energy reserves. The β-oxidation of fat stores of Lep−/− mice is not an option for fuel to maintain body temperature (37) and this is a major factor in cold intolerance of leptin-deficient mice during acute exposure (38). On the other hand, the wild-type mouse on a low fat chow diet can access its energy reserves in an acute situation, but quickly turns to increased food intake to maintain energy balance. This similarity in the metabolic response to the cold environment between normal and leptin-deficient mouse suggests that leptin is not important for the acute thermogenic phenotype in the Lep−/− mouse, nor for the regulation of food intake during cold exposure by normal wild-type mice fed a chow diet.

Mice with mutations to leptin and the leptin receptor have a thermogenic phenotype in which body temperature drops about 10° C. in about 4 hours at an ambient temperature of 4° C. (21); however, as illustrated in FIG. 3D they can adapt to the cold when it is gradually reduced. A key feature of cold-induced thermogenesis in normal animals is the increase in food intake that occurs over and above the increase in food intake necessary to support nutrition (39-42); as exemplified by the remarkable boost in food intake that occurs in lactating females exposed to the cold (33). This suggests that central mechanisms controlling food intake, as related to nutrition, growth and body composition, may be independent of those associated with cold-induced thermogenesis. A similar idea has been put forth by Speakman and Krol (43), but with a necessary role for leptin in the cold-induced food intake, which we did not see, nor was a role for leptin proposed by Melnyk and Himms-Hagen (6). The need to activate and fuel heat production virtually instantaneously when challenged by the cold is too vital for survival to wait for fat-derived leptin, unless there are packets of leptin parked in the hypothalamus for instantaneous release. We had observed previously, as did Coleman (20), that Lep−/− mice exposed to the cold further increased food intake above that normally occurring in these mice (19). This preliminary observation has been extended in this study to show that this hyperphagia, which is above that normally occurring in Lep−/− mice fed a chow diet, is very similar in magnitude and kinetics to that occurring in Lep+/+ mice. Accordingly, mechanisms controlling cold-associated food intake in Lep−/− mice are independent of leptin-based regulation of food intake. We tested further the role of leptin in regulating thermogenesis during cold exposure in wild-type DIO mice. Plasma leptin and insulin levels in DIO mice of this study are remarkably similar to mice described in previous studies that were leptin resistant (44). If the mobilization of fuels for cold-induced thermogenesis in DIO mice is controlled by the leptin resistance at the time of cold exposure, then one would predict that food intake would be high and mobilization of endogenous fat stores would be low. However, within one day of exposure to the cold the opposite phenotype was observed in DIO mice: food intake was low and fat mobilization was high. Even 4 days after cold exposure plasma levels of leptin were not significantly different from those at 24° C. (FIG. 2C); only after 7 days in the cold were the levels of leptin significantly reduced (FIG. 2D). This data additionally suggests that the mechanism controlling food intake during acute cold exposure is independent of leptin signaling. Chronic cold adaptation may involve leptin by another mechanism (19).

The primary motive driving this study was to explore the feasibility of using cold exposure as an anti-obesity strategy. Human studies on brown fat show dramatic inter-individual differences in brown adipocyte content and BAT activity (45, 46). Thus, it is important to assess how the cold-stimulated effect of body weight reduction is influenced if the capacity for thermogenesis in brown fat is variable. The extent to which BAT-mediated adaptive thermogenesis could account for variability in substrate utilization in the reduced ambient temperature is also not known. For these reasons we evaluated the phenotype of DIO Ucp1−/− mice lacking functional brown fat. Ucp1−/− mice are sensitive to the cold; however, they can adapt to the cold if the ambient temperature is gradually reduced (24, 47). Therefore, if UCP1 is essential to the thermogenic process, then in its absence the capacity for heat production from brown fat would be severely suppressed and we could expect effects on food intake and or the utilization of endogenous fuels that would differ from the wild-type mouse. As expected, when the ambient temperature was reduced average food intake was higher in the Ucp1−/− mice than in control mice, because these mice are less obese when fed a high-fat diet and they burned less of their endogenous reserves compared to normal Ucp1+/? mice with the greater obese phenotype (FIGS. 4B and 4D). Thus, there does not seem to be any difference in the pattern of utilization of endogenous food reserves or food intake between UCP1-deficient and wild type mice, provided that these mice have similar adiposity phenotypes as occurs with the lesser and greater obese mice.

QRT-PCR analysis of the expression of several genes in the hypothalamus encoding neuropeptides implicated with food intake did not provide strong evidence for the involvement of any of the neuropeptides associated with food intake with the possible exception of CART and POMC which have expression reduced by 30% and 100% in Lep+/+ only. However, a microarray analysis of gene expression in Lep−/− and Lep+/+ mice at 24 and 6° C. showed that the expression of neuropeptide VF precursor was decreased 4-fold during cold exposure in all three of the genetic models we have studied. In rodent brain, the sequence of the Npvf precursor gene predicts two -RFamide peptides: RFRP-1 and RFRP-3, also named NPSF and NPVF (48, 49). There is an expanding body of evidence for a role of various -RFamide peptides in the modulation of nociception, hormone secretion, reproduction or blood pressure (50-52). Finally, although little is known of the functional significance of this particular biological effect, various -RFamide peptides were able to illicit a transient 10-300% induction or suppression of food intake in chicks, rats or mice after i.c.v. injection (50, 53-56). Moreover, food restriction or deprivation, both stimulating hunger and food hoarding, have been shown to be positively correlated with activation of RFRP-3 cells in the DMH of Syrian hamsters (57). Effects on thermogenesis are unknown. From a functional viewpoint, specific expression of Npvf mRNA in the rodent central nervous system is restricted to a population of neurons localized between dorsomedial hypothalamic (DMH) and ventromedial hypothalamic (VMH) nucleus (48, 49, 58, 59), which is consistent with a putative role in feeding or thermogenic processes (60). Finally the microarray study showed that the cold environment induces the components of the dopamine signaling cascade in both Lep−/− and Lep+/+ mice. The dopaminergic system is involved in a variety of critical functions among them feeding behavior and is disrupted in eating disorders such as anorexia or obesity (29, 61). There is increasing evidence that the neuropeptides regulating energy balance through the hypothalamus can also modulate the activity of dopamine expressing neurons and their projections into extra-hypothalamic brain regions involved in the rewarding processes of food stimuli (29, 62). A thermoregulatory role for vasopressin is well documented (63, 64), therefore the observation that reduced ambient temperature modulates expression of Avp mRNA in hypothalamus is not surprising. While the one hand AVP can affect body temperature by acting in the periphery on blood vessel contraction and on the other hand, there is evidence that AVP-containing fibers are involved in the central control of the thermoregulatory system by modulating firing activity of thermosensitive neurons localized in the preoptic-anterior area of hypothalamus in rats (65, 66) and rabbits (67).

In conclusion, an analysis of three mouse models of obesity suggests that reduced ambient temperature is effective in reducing diet-induced obesity without long-term compensatory increases in food intake. This conclusion is based in part on the finding cold-activated thermogenesis is primarily fueled by endogenous fat stores in animals with diet-induced obesity. We also identify Npvf, which encodes a novel RFamide-related peptide, as a novel biomarker of cold-induced thermogenesis in the hypothalamus. Changes in expression of Npvf are activated by the cold in a manner that is independent of nutritional status.

### REFERENCES

1. Seale, P., and Lazar, M. A. 2009. Brown fat in humans: turning up the heat on obesity. Diabetes 58:1482-1484.
2. Caudwell, P., Gibbons, C., Hopkins, M., Naslund, E., King, N., Finlayson, G., and Blundell, J. 2011. The influence of physical activity on appetite control: an experimental system to understand the relationship between exercise-induced energy expenditure and energy intake. Proc Nutr Soc 70:171-180.
3. Kennedy, G. C. 1953. The role of depot fat in the hypothalamic control of food intake in the rat. Proc R Soc Lond B Biol Sci 140:578-596.
4. Cannon, B., and Nedergaard, J. 2009. Thermogenesis challenges the adipostat hypothesis for body-weight control. Proc Nutr Soc 68:401-407.
5. Arch, J. R. 2008. The discovery of drugs for obesity, the metabolic effects of leptin and variable receptor pharmacology: perspectives from beta3-adrenoceptor agonists. Naunyn Schmiedebergs Arch Pharmacol 378:225-240.
6. Melnyk, A., and Himms-Hagen, J. 1998. Temperature-dependent feeding: lack of role for leptin and defect in brown adipose tissue-ablated obese mice. Am J Physiol 274:R1131-1135.
7. Perello, M., Stuart, R. C., Vaslet, C. A., and Nillni, E. A. 2007. Cold exposure increases the biosynthesis and proteolytic processing of prothyrotropin-releasing hormone in the hypothalamic paraventricular nucleus via beta-adrenoreceptors. Endocrinology 148:4952-4964.
8. Park, J. J., Lee, H. K., Shin, M. W., Kim, S. J., Noh, S. Y., Shin, J., and Yu, W. S. 2007. Short-term cold exposure may cause a local decrease of neuropeptide Y in the rat hypothalamus. Mol Cells 23:88-93.
9. Cabral, A., Valdivia, S., Reynaldo, M., Cyr, N. E., Nillni, E. A., and Perello, M. 2012. Short-term cold exposure activates TRH neurons exclusively in the hypothalamic paraventricular nucleus and raphe pallidus. Neurosci Lett 518:86-91.
10. Pereira-da-Silva, M., Torsoni, M. A., Nourani, H. V., Augusto, V. D., Souza, C. T., Gasparetti, A. L., Carvalheira, J. B., Ventrucci, G., Marcondes, M. C., Cruz-Neto, A. P., et al. 2003. Hypothalamic melanin-concentrating hormone is induced by cold exposure and participates in the control of energy expenditure in rats. Endocrinology 144:4831-4840.
11. Sanchez, E., Fekete, C., Lechan, R. M., and Joseph-Bravo, P. 2007. Cocaine- and amphetamine-regulated transcript (CART) expression is differentially regulated in the hypothalamic paraventricular nucleus of lactating rats exposed to suckling or cold stimulation. Brain Res 1132:120-128.
12. McCarthy, H. D., Kilpatrick, A. P., Trayhurn, P., and Williams, G. 1993. Widespread increases in regional hypothalamic neuropeptide Y levels in acute cold-exposed rats. Neuroscience 54:127-132.
13. Egawa, M., Yoshimatsu, H., and Bray, G. A. 1991. Neuropeptide Y suppresses sympathetic activity to interscapular brown adipose tissue in rats. Am J Physiol 260:R328-334.
14. Small, C. J., Liu, Y. L., Stanley, S. A., Connoley, I. P., Kennedy, A., Stock, M. J., and Bloom, S. R. 2003. Chronic CNS administration of Agouti-related protein (Agrp) reduces energy expenditure. Int J Obes Relat Metab Disord 27:530-533.
15. Chao, P. T., Yang, L., Aja, S., Moran, T. H., and Bi, S. 2011. Knockdown of NPY expression in the dorsomedial hypothalamus promotes development of brown adipocytes and prevents diet-induced obesity. Cell Metab 13:573-583.
16. Dimitrov, E. L., Kim, Y. Y., and Usdin, T. B. 2011. Regulation of hypothalamic signaling by tuberoinfundibular peptide of 39 residues is critical for the response to cold: a novel peptidergic mechanism of thermoregulation. J Neurosci 31:18166-18179.
17. Nillni, E. A., Xie, W., Mulcahy, L., Sanchez, V. C., and Wetsel, W. C. 2002. Deficiencies in pro-thyrotropin-releasing hormone processing and abnormalities in thermoregulation in Cpefat/fat mice. J Biol Chem 277:48587-48595.
18. Koza, R. A., Nikonova, L., Hogan, J., Rim, J. S., Mendoza, T., Faulk, C., Skaf, J., and Kozak, L. P. 2006. Changes in gene expression foreshadow diet-induced obesity in genetically identical mice. PLoS Genet 2:e81.
19. Ukropec, J., Anunciado, R. V., Ravussin, Y., and Kozak, L. P. 2006. Leptin is required for uncoupling protein-1-independent thermogenesis during cold stress. Endocrinology 147:2468-2480.
20. Coleman, D. L. 1982. Thermogenesis in diabetes-obesity syndromes in mutant mice. Diabetologia 22:205-211.
21. Trayhurn, P., and James, W. P. 1978. Thermoregulation and non-shivering thermogenesis in the genetically obese (ob/ob) mouse. Pflugers Arch 373:189-193.
22. Enerback, S., Jacobsson, A., Simpson, E. M., Guerra, C., Yamashita, H., Harper, M. E., and Kozak, L. P. 1997. Mice lacking mitochondrial uncoupling protein are cold-sensitive but not obese. Nature 387:90-94.
23. Liu, X., Rossmeisl, M., McClaine, J., Riachi, M., Harper, M. E., and Kozak, L. P. 2003. Paradoxical resistance to diet-induced obesity in UCP1-deficient mice. J Clin Invest 111:399-407.
24. Golozoubova, V., Hohtola, E., Matthias, A., Jacobsson, A., Cannon, B., and Nedergaard, J. 2001. Only UCP1 can mediate adaptive nonshivering thermogenesis in the cold. FASEB J 15:2048-2050.
25. Brobeck, J. R. 1948. Food intake as a mechanism of temperature regulation. Yale J Biol Med 20:545-552.
26. Ukropec, J., Anunciado, R. P., Ravussin, Y., Hulver, M. W., and Kozak, L. P. 2006. UCP1-independent thermogenesis in white adipose tissue of cold-acclimated Ucp1−/− mice. J Biol Chem 281:31894-31908.
27. Befort, K., Filliol, D., Ghate, A., Darcq, E., Matifas, A., Muller, J., Lardenois, A., Thibault, C., Dembele, D., Le Merrer, J., et al. 2008. Mu-opioid receptor activation induces transcriptional plasticity in the central extended amygdala. Eur J Neurosci 27:2973-2984.
28. Grobin, A. C., Matthews, D. B., Devaud, L. L., and Morrow, A. L. 1998. The role of GABA(A) receptors in the acute and chronic effects of ethanol. Psychopharmacology (Berl) 139:2-19.
29. Baik, J. H. 2013. Dopamine signaling in reward-related behaviors. Front Neural Circuits 7:152.
30. Wu, P., and Childs, G. V. 1990. Cold and novel environment stress affects AVP mRNA in the paraventricular nucleus, but not the supraoptic nucleus: An in Situ hybridization study. Mol Cell Neurosci 1:233-249.
31. Angulo, J. A., Ledoux, M., and McEwen, B. S. 1991. Genomic effects of cold and isolation stress on magnocellular vasopressin mRNA-containing cells in the hypothalamus of the rat. J Neurochem 56:2033-2038.
32. Rizwan, M. Z., Harbid, A. A., Inglis, M. A., Quennell, J. H., and Anderson, G. M. 2014. Evidence that hypothalamic RFamide related peptide-3 neurones are not leptin-responsive in mice and rats. J Neuroendocrinol 26:247-257.
33. Johnson, M. S., and Speakman, J. R. 2001. Limits to sustained energy intake. V. Effect of cold-exposure during lactation in Mus musculus. J Exp Biol 204:1967-1977.
34. Melzer, K., Kayser, B., Saris, W. H., and Pichard, C. 2005. Effects of physical activity on food intake. Clin Nutr 24:885-895.
35. Leibel, R. L., Rosenbaum, M., and Hirsch, J. 1995. Changes in energy expenditure resulting from altered body weight. N Engl J Med 332:621-628.
36. Bukowiecki, L. J. 1989. Energy balance and diabetes. The effects of cold exposure, exercise training, and diet composition on glucose tolerance and glucose metabolism in rat peripheral tissues. Can J Physiol Pharmacol 67:382-393.
37. Minokoshi, Y., Kim, Y. B., Peroni, O. D., Fryer, L. G., Muller, C., Carling, D., and Kahn, B. B. 2002. Leptin stimulates fatty-acid oxidation by activating AMP-activated protein kinase. Nature 415:339-343.
38. Guerra, C., Koza, R. A., Walsh, K., Kurtz, D. M., Wood, P. A., and Kozak, L. P. 1998. Abnormal nonshivering thermogenesis in mice with inherited defects of fatty acid oxidation. J Clin Invest 102:1724-1731.
39. Harris, R. B., Mitchell, T. D., Kelso, E. W., and Flatt, W. P. 2007. Changes in environmental temperature influence leptin responsiveness in low- and high-fat-fed mice. Am J Physiol Regul Integr Comp Physiol 293:R106-115.
40. Bing, C., Frankish, H. M., Pickavance, L., Wang, Q., Hopkins, D. F., Stock, M. J., and Williams, G. 1998. Hyperphagia in cold-exposed rats is accompanied by decreased plasma leptin but unchanged hypothalamic NPY. Am J Physiol 274:R62-68.
41. Zhao, Z. J. 2011. Serum leptin, energy budget, and thermogenesis in striped hamsters exposed to consecutive decreases in ambient temperatures. Physiol Biochem Zool 84:560-572.
42. Krol, E., and Speakman, J. R. 2003. Limits to sustained energy intake. VI. Energetics of lactation in laboratory mice at thermoneutrality. J Exp Biol 206:4255-4266.
43. Speakman, J. R., and Krol, E. 2011. Limits to sustained energy intake. XIII. Recent progress and future perspectives. J Exp Biol 214:230-241.
44. El-Haschimi, K., Pierroz, D. D., Hileman, S. M., Bjorbaek, C., and Flier, J. S. 2000. Two defects contribute to hypothalamic leptin resistance in mice with diet-induced obesity. J Clin Invest 105:1827-1832.
45. Yoneshiro, T., Aita, S., Matsushita, M., Okamatsu-Ogura, Y., Kameya, T., Kawai, Y., Miyagawa, M., Tsujisaki, M., and Saito, M. 2011. Age-related decrease in cold-activated brown adipose tissue and accumulation of body fat in healthy humans. Obesity (Silver Spring) 19:1755-1760.
46. Cypess, A. M., Lehman, S., Williams, G., Tal, I., Rodman, D., Goldfine, A. B., Kuo, F. C., Palmer, E. L., Tseng, Y. H., Doria, A., et al. 2009. Identification and importance of brown adipose tissue in adult humans. N Engl J Med 360:1509-1517.
47. Hofmann, W. E., Liu, X., Bearden, C. M., Harper, M. E., and Kozak, L. P. 2001. Effects of genetic background on thermoregulation and fatty acid-induced uncoupling of mitochondria in UCP1-deficient mice. J Biol Chem 276:12460-12465.
48. Liu, Q., Guan, X. M., Martin, W. J., McDonald, T. P., Clements, M. K., Jiang, Q., Zeng, Z., Jacobson, M., Williams, D. L., Jr., Yu, H., et al. 2001. Identification and characterization of novel mammalian neuropeptide FF-like peptides that attenuate morphine-induced antinociception. J Biol Chem 276:36961-36969.
49. Hinuma, S., Shintani, Y., Fukusumi, S., Iijima, N., Matsumoto, Y., Hosoya, M., Fujii, R., Watanabe, T., Kikuchi, K., Terao, Y., et al. 2000. New neuropeptides containing carboxy-terminal RFamide and their receptor in mammals. Nat Cell Biol 2:703-708.
50. Johnson, M. A., Tsutsui, K., and Fraley, G. S. 2007. Rat RFamide-related peptide-3 stimulates GH secretion, inhibits LH secretion, and has variable effects on sex behavior in the adult male rat. Horm Behav 51:171-180.
51. Yang, H. Y., Fratta, W., Majane, E. A., and Costa, E. 1985. Isolation, sequencing, synthesis, and pharmacological characterization of two brain neuropeptides that modulate the action of morphine. Proc Natl Acad Sci USA 82:7757-7761.
52. Jhamandas, J. H., and Goncharuk, V. 2013. Role of neuropeptide FF in central cardiovascular and neuroendocrine regulation. Front Endocrinol (Lausanne) 4:8.
53. Tachibana, T., Sato, M., Takahashi, H., Ukena, K., Tsutsui, K., and Furuse, M. 2005. Gonadotropin-inhibiting hormone stimulates feeding behavior in chicks. Brain Res 1050:94-100.
54. Cline, M. A., Bowden, C. N., Calchary, W. A., and Layne, J. E. 2008. Short-term anorexigenic effects of central neuropeptide VF are associated with hypothalamic changes in chicks. J Neuroendocrinol 20:971-977.
55. Chartrel, N., Dujardin, C., Anouar, Y., Leprince, J., Decker, A., Clerens, S., Do-Rego, J. C., Vandesande, F., Llorens-Cortes, C., Costentin, J., et al. 2003. Identification of 26RFa, a hypothalamic neuropeptide of the RFamide peptide family with orexigenic activity. Proc Natl Acad Sci USA 100:15247-15252.
56. Murakami, M., Matsuzaki, T., Iwasa, T., Yasui, T., Irahara, M., Osugi, T., and Tsutsui, K. 2008. Hypophysiotropic role of RFamide-related peptide-3 in the inhibition of LH secretion in female rats. J Endocrinol 199:105-112.
57. Klingerman, C. M., Williams, W. P., 3rd, Simberlund, J., Brahme, N., Prasad, A., Schneider, J. E., and Kriegsfeld, L. J. 2011. Food Restriction-Induced Changes in Gonadotropin-Inhibiting Hormone Cells are Associated with Changes in Sexual Motivation and Food Hoarding, but not Sexual Performance and Food Intake. Front Endocrinol (Lausanne) 2:101.
58. Yano, T., Iijima, N., Kakihara, K., Hinuma, S., Tanaka, M., and Ibata, Y. 2003. Localization and neuronal response of RFamide related peptides in the rat central nervous system. Brain Res 982:156-167.
59. Kriegsfeld, L. J., Mei, D. F., Bentley, G. E., Ubuka, T., Mason, A. O., Inoue, K., Ukena, K., Tsutsui, K., and Silver, R. 2006. Identification and characterization of a gonadotropin-inhibitory system in the brains of mammals. Proc Natl Acad Sci USA 103:2410-2415.
60. Morrison, S. F., Madden, C. J., and Tupone, D. 2014. Central Neural Regulation of Brown Adipose Tissue Thermogenesis and Energy Expenditure. Cell Metab.
61. Berridge, K. C., Ho, C. Y., Richard, J. M., and DiFeliceantonio, A. G. 2010. The tempted brain eats: pleasure and desire circuits in obesity and eating disorders. Brain Res 1350:43-64.
62. Figlewicz, D. P. 2003. Adiposity signals and food reward: expanding the CNS roles of insulin and leptin. Am J Physiol Regul Integr Comp Physiol 284:R882-892.
63. Raggenbass, M. 2008. Overview of cellular electrophysiological actions of vasopressin. Eur J Pharmacol 583:243-254.
64. Richmond, C. A. 2003. The role of arginine vasopressin in thermoregulation during fever. J Neurosci Nurs 35:281-286.
65. Tang, Y., Yang, Y. L., Wang, N., Shen, Z. L., Zhang, J., and Hu, H. Y. 2012. Effects of arginine vasopressin on firing activity and thermosensitivity of rat PO/AH area neurons. Neuroscience 219:10-22.
66. Moravec, J., and Pierau, F. K. 1994. Arginine-Vasopressin Modifies the Firing Rate and Thermosensitivity of Neurons in Slices of the Rat Po/Ah Area. Integrative and Cellular Aspects of Autonomic Functions: Temperature and Osmoregulation:143-152.
67. Yang, Y. L., and Chen, B. Y. 1994. The effect of microinjection of AVP and AVP-antiserum into septal area on discharge activity of temperature-sensitive neurons in PO-AH of rabbits. Sheng Li Xue Bao 46:141-147.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one or all of the group members are present in, employed in or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

We claim:

1. A method for promoting energy utilization in a subject, comprising
administering to the subject an effective amount of a Npvf pathway inhibitor to promote energy utilization.

2. The method of claim 1, wherein the Npvf pathway inhibitor is an Npvf pathway antagonist.

3. The method of claim 2, wherein the Npvf pathway antagonist is an anti-RFRP3 antibody or an anti-RFRP1 antibody.

4. The method of claim 2, wherein the Npvf pathway antagonist is RF9 or BIBP3226.

5. The method of claim 1, wherein the Npvf pathway inhibitor is an expression inhibitor.

6. The method of claim 5, wherein the expression inhibitor is an inhibitory nucleic acid.

7. The method of claim 6, wherein the inhibitory nucleic acid is an antisense oligonucleotide.

8. The method of claim 6, wherein the inhibitory nucleic acid is a siRNA.

9. The method of claim 1, wherein the Npvf pathway inhibitor is administered orally.

10. The method of claim 1, wherein the Npvf pathway inhibitor is administered by injection.

11. The method of claim 1 further comprising administering to the subject a dopamine antagonist.

12. A method for treating a wasting disorder in a subject, comprising
administering to the subject an effective amount of a Npvf pathway inducing agent to reduce energy utilization.

13. The method of claim 12, wherein the wasting disorder is anorexia nervosa.

14. The method of claim 12, wherein the Npvf pathway inducing agent is exogenous Npvf.

15. The method of claim 14, wherein the exogenous Npvf comprises RFRP1.

16. The method of claim 14, wherein the exogenous Npvf comprises RFRP3.

17. The method of claim 14, wherein the exogenous Npvf comprises RFRP1 and RFPR3.

18. The method of claim 12, wherein the Npvf pathway inducing agent is an Npvf expression vector.

19. The method of claim 12, wherein the Npvf pathway inducing agent is an Npvf mRNA.

20. The method of claim 12, wherein the Npvf pathway inducing agent is an Npvf activator selected that is melatonin.

21. The method of claim 12, wherein the Npvf pathway inducing agent is an NPFF-R1 agonist.

22. The method of claim 12, wherein the Npvf pathway inducing agent is an NPFF-R2 agonist.

23. The method of claim 12, further comprising administering to the subject a dopaminergic pathway antagonist.

24. The method of claim 12, wherein the Npvf pathway inducing agent is administered orally.

25. The method of claim 12, wherein the Npvf pathway inducing agent is administered by injection.
